SEQUENCE LISTING (25 sequences)

SEQ ID NO: 1 - protein, 18 aa, Thosea asigna virus
EGRGSLLTCGDVEENPGP

SEQ ID NO: 2 - DNA, 20 bp, Artificial Sequence
  target sequence A1
gtgaagacatcccccactgt

SEQ ID NO: 3 - DNA, 20 bp, Artificial Sequence
  target sequence A2
tgtgaagacatcccccactg

SEQ ID NO: 4 - DNA, 20 bp, Artificial Sequence
  target sequence B1
ggcaagatgagatatttatg

SEQ ID NO: 5 - DNA, 20 bp, Artificial Sequence
  target sequence B2
aagatgagatatttatgtgg

SEQ ID NO: 6 - DNA, 50 bp, Artificial Sequence
  Bxb1 attB site
tcggccggcttgtcgacgacggcggtctccgtcgtcaggatcatccgggc

SEQ ID NO: 7 - DNA, 58 bp, Artificial Sequence
  Bxb1 attP site
gtcgtggtttgtctggtcaaccaccgcggtctcagtggtgtacggtacaaaccccgac

SEQ ID NO: 8 - DNA, 54 bp, Artificial Sequence
  Bxb1 attL site
tcggccggcttgtcgacgacggcggtctcagtggtgtacggtacaaaccccgac

SEQ ID NO: 9 - DNA, 54 bp, Artificial Sequence
  Bxb1 attR site
gtcgtggtttgtctggtcaaccaccgcggtctccgtcgtcaggatcatccgggc

SEQ ID NO: 10 - DNA, 45 bp, Artificial Sequence
  PhiC31 attB site
tgcgggtgccagggcgtgcccttgggctccccgggcgcgtactcc

SEQ ID NO: 11 - DNA, 42 bp, Artificial Sequence
  PhiC31 attP site
gtgccccaactggggtaacctttgagttctctcagttggggg

SEQ ID NO: 12 - DNA, 42 bp, Artificial Sequence
  PhiC31 attL site
tgcgggtgccagggcgtgcccttgagttctctcagttggggg

SEQ ID NO: 13 - DNA, 45 bp, Artificial Sequence
  PhiC31 attR site
gtgccccaactggggtaacctttgggctccccgggcgcgtactcc

SEQ ID NO: 14 - DNA, 20 bp, Artificial Sequence
  PuroR rev primer
gagttcttgcagctcggtga

SEQ ID NO: 15 - DNA, 19 bp, Artificial Sequence
  tdTomato 3′ Rev primer
ggcatggacgagctgtata

SEQ ID NO: 16 - DNA, 20 bp, Artificial Sequence
  F2 MAPT AS primer
ggactgaggtgggggtgata

SEQ ID NO: 17 - DNA, 25 bp, Artificial Sequence
  SV40 Poly A Rev primer
gctttatttgtgaaatttgtgatgc

SEQ ID NO: 18 - DNA, 22 bp, Artificial Sequence
  Kansl Insertion Assay R1 primer
gcacatcatctgggagagacta

SEQ ID NO: 19 - DNA, 19 bp, Artificial Sequence
  Hygro to 3′ arm F2 primer
cagacgcgctacttcgagc

SEQ ID NO: 20 - DNA, 22 bp, Artificial Sequence
  Kansl Insertion Assay R1 primer
gcacatcatctgggagagacta

SEQ ID NO: 21 - DNA, 20 bp, Artificial Sequence
  F2 MAPT AS primer
ggactgaggtgggggtgata

SEQ ID NO: 22 - DNA, 22 bp, Artificial Sequence
  3′ of MAPT-AS primer
agtcagttggcagtttggatga

SEQ ID NO: 23 - DNA, 20 bp, Artificial Sequence
  Primer B
acgagccttcatagcatccg

SEQ ID NO: 24 - DNA, 21 bp, Artificial Sequence
  Hygro connect primer
tccacgccctcctacatcgaa

SEQ ID NO: 25 - DNA, 20 bp, Artificial Sequence
  MAPT-KANSL F primer
taggtttgcagagctgcctt

(19) United States
(12) Patent Application Publication
(10) Pub. No.: US 2020/0109422 A1
(43) Pub. Date: Apr. 9, 2020

(54) METHODS OF FULL GENE REPLACEMENT AND TRANSGENIC NON-HUMAN CELLS COMPRISING FULL HUMAN GENES

(71) Applicant: Regents of the University of Minnesota, Minneapolis, MN (US)

(72) Inventors: Michael Koob, Minneapolis, MN (US); Kellie Benzow, Minneapolis, MN (US)

(21) Appl. No.: 16/596,228

(22) Filed: Oct. 8, 2019

Related U.S. Application Data
(60) Provisional application No. 62/743,214, filed on Oct. 9, 2018.

(51) Int. Cl. C12N 15/90; A01K 67/027

(52) U.S. Cl. CPC C12N 15/907; A01K 67/0278; C12N 2310/20; A01K 2227/105; A01K 2207/15; A01K 2217/072; C12N 2800/80

(57) ABSTRACT

Provided herein are precise gene replacement methods and transgenic non-human animals produced by such methods, in which an endogenous non-human animal gene of interest is precisely replaced with a human syntenic gene. The resulting genetically modified non-human animals are useful for evaluating molecular impact of pathogenic mutations within the context of the human genomic sequence in which they occur in patients and for screening for potential therapeutic agents.

### CROSS-REFERENCE TO RELATED APPLICATIONS

This application claims the benefit of U.S. Provisional Application No. 62/743,214, filed Oct. 9, 2018, the disclosure of which is hereby incorporated by reference in its entirety for all purposes.

### STATEMENT REGARDING FEDERALLY FUNDED RESEARCH OR DEVELOPMENT

Not applicable.

### BACKGROUND

One of the major unexpected findings of the Human Genome project is that humans evolved from our much less complex ancestors by modifying the regulation of a relatively small number of genes in increasingly complex ways. Waves of carefully orchestrated, temporally- and spatially-differentiated expression of a range of protein variants encoded in alternatively spliced messenger isoforms establish and maintain all of our diverse tissues and cell types and all of their varied functions. We can readily predict the sequence of the proteins written in the minor “coding” portions of our genes, but we are just learning to understand and appreciate the full complexity of what is written in the “non-coding” sequences that comprise the bulk of our genes.

Standard tools of molecular genetics are best adapted for working with relatively small DNA fragments, and as a result most of the animal models generated by researchers and currently available to the wider biomedical research community incorporate short, synthetic and very simplified cDNA versions of genes expressed from short, exogenous promoter fragments. Indeed, typical homologous recombination approaches for use in animal cells (for example, embryonic stem (ES) cells or embryos) are generally not suitable for replacing more than a few kilobases (kb) of the animal genomic sequence since the DNA to be inserted recombines at unexpected locations. Genomic position effects on transgene expression patterns as well as gene disruption and dysregulation caused by random insertion events invariably result in dramatic phenotypic variation that can easily mask phenotypic changes that might result from small changes in transgene sequence. Other attempts to introducing syntenic portions of a human genome into an animal genome involved large deletions and large scale modifications. Accordingly, there remains a need in the field for targeted methods for precise replacement of a gene in non-human animal cells with a syntenic human gene.

### SUMMARY OF THE PRESENT DISCLOSURE

In a first aspect, provided herein is a method for removing an endogenous gene in the genome of a non-human animal, the method comprising: introducing into a non-human animal cell: (a) a first construct comprising (i) a nucleic acid sequence encoding a first selectable marker, the nucleic acid sequence flanked by a first recombination site and a second recombination site, (ii) a promoter, and (iii) a nucleic acid sequence encoding a first recombinase, wherein components (ii) and (iii) are separated by a third recombination site, wherein the first, second, and third recombination sites do not recombine with each other, whereby the first selectable marker and the recombinase protein products are expressed by a single open reading frame, (b) a second construct comprising a nucleic acid sequence encoding a second selectable marker flanked by a fourth recombination site and a fifth recombination site, wherein the fourth recombination site is upstream of the second selectable marker and is capable of recombining with the third recombination site in the presence of the first recombinase; (c) one or more Cas9/gRNA constructs comprising one or more gRNAs having sequence complementary to at least a portion of the endogenous non-human gene, wherein gRNAs expressed from the one or more Cas9/gRNA constructs associate with the endogenous target gene and generate double stranded breaks 5′ and 3′ to the endogenous non-human gene, wherein the double stranded breaks are repaired by homology-directed repair using the first and second constructs, whereby the first and second constructs are inserted 5′ and 3′ to the endogenous non-human target gene, and wherein expression of the recombinase from the first construct catalyzes recombination between the third and fourth recombination sites, whereby the endogenous non-human gene and the nucleic acid encoding the recombinase are excised and the promoter drives expression of the selectable marker. The first targeting construct can further comprise, located between the third recombination site and the first recombinase-encoding sequence, a nucleic acid sequence encoding a peptide separation linker. The method can further comprise selecting a non-human cell in which the endogenous non-human gene is excised by detecting expression of the selectable marker and the screening marker. The first or second selectable marker can be a fluorescent marker. The first or second selectable marker can be a drug resistance marker. The third and fourth recombination sites can be lox recombination sites and wherein the site-specific recombinase is Cre.

In another aspect, provided herein is a method for replacing an endogenous gene in the genome of a non-human animal with a syntenic sequence from a human genome, the method comprising obtaining a non-human cell in which an endogenous gene has been excised according to a method described herein; introducing into the obtained cell a nucleic acid vector comprising nucleic acid sequence syntenic to the excised endogenous gene, where the syntenic sequence is flanked on the 5′ end by a sixth recombination site and flanked on the 3′ end by a seventh recombination site, the vector further comprising a promoter operably linked to a nucleic acid sequence encoding a second recombinase and an eighth recombination site, where the fifth and seventh recombination sites recombine with each other, and where the second and eighth recombination sites recombine with each other in the presence of the second recombinase; wherein expression of the first recombinase catalyzes recombination between the first and sixth recombination sites, resulting in insertion of the syntenic sequence in place of the excised endogenous non-human gene, and wherein expression of the second recombinase catalyzes recombination between the fifth and seventh recombination sites and the second and eighth recombination sites to excise nucleic acid sequences encoding the screening marker and the selection marker. The second and eighth recombination sites can be FRT recombination sites, and wherein the fifth and seventh recombination sites can be FRT3 recombination sites. The nucleic acid vector can comprise a PGK promoter.

In another aspect, provided herein is a genetically modified non-human cell comprising a human syntenic gene generated by a method of this disclosure. The non-human animal cell can be a mouse embryonic cell.

In a further aspect, provided herein is a genetically modified non-human animal generated from a genetically modified of this disclosure. The animal can be a mammal. The animal can be chosen from a mouse, a rat, a rabbit, a pig, a sheep, a goat, poultry, and a cow.

These and other embodiments, aspects, advantages, and features of the present invention will be set forth in part in the description which follows, and will become apparent to those skilled in the art by reference to the following description of the invention and referenced drawings or by practice of the invention. The accompanying drawings illustrate one or more implementations, and these implementations do not necessarily represent the full scope of the invention.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, where:

FIGS. 1A-1B illustrate an embodiment of the gene replacement methods described herein for replacement of TCF4. (A) One-step deletion of a 143 kb segment of the mouse Tcf4 gene in mouse ES cells. (B) Flp-in of the 191 kb syntenic segment of the human TCF4 gene, followed by deletion of the final selection cassette.

FIG. 2 illustrates proof-of concept models for human gene replacement. A 191 kb segment of the human TCF4 gene (larger, blue line) has precisely replaced the 143 kb syntenic segment of the mouse Tcf4 gene. The control line has a wild-type (wt) repeat (24xCTG) and the matched experimental line has an expanded pathogenic trinucleotide repeat (83xCTG, red arrow).

FIG. 3 is an image showing the result of ES cell junction PCR assays following gel electrophoresis.

FIG. 4 illustrates an embodiment of the gene replacement methods provided herein for replacement of MAPT. A 157 kb segment of the Mapt gene was replaced in mouse ES cells with a “Flp-in” cassette, and use Flp recombinase to integrate human MAPT constructs (190 kb) at this site. The selection cassette has been removed in a subsequent breeding step by CRE recombinase.

FIGS. 5A-5B demonstrate complete replacement of the mouse Mapt gene with the human MAPT gene. (A) Genotyping data from a litter of mice carrying the complete human MAPT (H2) gene in place of the mouse Mapt gene (mice positive for the human MAPT gene replacement are starred). The insertion junctions between the human and mouse sequences (shown) as well as several internal segments (not shown) were amplified and sequenced, and the complete integrity of this segment in the resulting mouse line will be confirmed by whole-genome sequencing once we obtain lines homozygous for the MAPT allele (next generation). m=marker, ES=DNA from ES cell control. (B) Human-specific MAPT mRNA reverse-transcription PCR (rtPCR) analyses were performed on forebrain RNA isolated from the first two mice in this litter (in the example shown, we amplified mRNA sequences from the sixth marked exon to the last). Preliminary sequence analyses of rtPCR products have confirmed correct splicing at the expected exon-exon junctions.

FIGS. 6A-6C demonstrate complete replacement of the mouse Mapt gene with the human MAPT gene. A) We precisely replaced the 157 kb mouse Mapt gene with the 190 kb human syntenic genomic segment encoding the complete MAPT coding region and MAPT-AS1 regulatory region (in green), as confirmed by NGS whole genome sequencing of mice homozygous for this MAPT-Gene Replacement (MAPT-GR) allele. The exons in the MAPT mRNA and in the long-noncoding antisense RNA transcripts are indicated, and flanking mouse genes are shown in black (size scale in kilobases). B) Western analyses show that human tau protein is expressed at endogenous levels in MAPT-GR mice. Equal amounts (50 μg) of protein isolated from the forebrain tissue of either a mouse homozygous for the transplanted MAPT-GR allele (GR) or from a wt C57BL/6 mouse (wt) were analyzed with an antibody (TAU-5) that binds equally well to either human or mouse tau protein. The equal intensity of signal from both sample shows that the human tau protein is expressed at levels equivalent to that of mouse tau. Analyses with an antibody that only recognizes human tau demonstrate that the tau expressed in the MAPT-GR mice is human (the bands in the mouse lane are non-tau background recognized by this antibody, also present in the MAPT-GR sample). C) Transcription analyses show that all of the human MAPT splice isoforms are expressed in the tissues and in the ratios expected for the human MAPT gene. RNA isolated from the cerebellum (Cer.) forebrain (F.B.), heart (Hrt.), and kidney (K.) of MAPT-GR homozygous mice, as well as from the forebrain of a wt C57BL/6 control animal (wt) were analyzed by rtPCR. We found MAPT transcripts that encoded either “0N” (no exon 2 or 3), “1N” (exon 2 but not 3), and “2N” (both exon 2 and 3) tau (first panel). We also found roughly equal levels of “3R” (no exon 10) and “4R” (with exon 10) MAPT transcripts in brain tissues, with different ratios in the other tissues examined, as is typical for the human MAPT gene (human-specific rtPCR panel). We did not detect any mouse Mapt transcripts in the MAPT-GR homozygote samples using mouse-specific rtPCR, but found the expected predominance of “4R” mouse tau transcript in the forebrain sample from the adult wt control animal.

FIG. 7 illustrates replacement of the mouse App gene (290 kb) with the human APP gene (356 kb). The APP sense and antisense transcribed regions are shown as gray arrows, with the segment of the human genome included in the APP-GR allele as gray line (flanking sequences not included are in black). Human-specific rtPCR analyses indicate that the human APP gene in this line is properly transcribed and processed (cDNA sequencing matched the human mRNA control perfectly, with no rtPCR signal from a wt mouse).

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION OF THE PRESENT DISCLOSURE

The methods and compositions provided herein are based, at least in part, on the inventors' development of a set of tools and methodologies that allow replacement of mouse genes with their full human orthologs, up to several hundred kilobases (kb) or more in size. The gene replacement methods and compositions described herein are particularly advantageous as the inventors made the surprising determination that CRISPR/Cas9 cleavage is not effective in removing large segments of a targeted genome. Advantageously, these gene replacement methods enable one to vastly expand the size of transgene constructs that can be precisely spliced into target genomes on a routine basis. Precisely matched sets of animal models developed using this approach will allow the research community to evaluate the molecular impact of pathogenic mutations within the context of the human genomic sequence in which they occur in patients, and these mouse lines will contain all of the potential human therapeutic targets ranging from the full genomic DNA sequences to all of the RNA transcription variants and protein products that they encode. Furthermore, because the genomic sequences of these matched sets will differ only at sequences specifically changed in each line, any significant molecular differences between these lines can be confidently attributed to the pathogenic mutation in the experimental lines, and any therapeutic agents found to effectively correct these dysfunctions could be expected to have direct therapeutic value to patients.

Accordingly, in a first aspect, provided herein are methods for removing an endogenous gene in the genome of a non-human animal and replacing the excised endogenous gene with a syntenic human gene. In a first step, the method comprises introducing into a non-human animal cell a first targeting construct and a second targeting construct. These targeting constructs provide recombination site useful for excising the endogenous gene. As used herein, the term “endogenous” refers to any material from or produced inside an organism, cell, tissue or system. As used herein, the term “exogenous” refers to any material introduced from or produced outside an organism, cell, tissue or system.

In some cases, the first targeting construct comprises (i) a nucleic acid sequence encoding a first selectable marker, the nucleic acid sequence flanked by a first recombination site and a second recombination site, (ii) a promoter, and (iii) a nucleic acid sequence encoding a first recombinase, wherein components (ii) and (iii) are separated by a third recombination site and, optionally, a nucleic acid sequence encoding a detectable marker, wherein the first, second, and third recombination sites do not recombine with each other. The first targeting construct can further comprise, located between the third recombination site and the recombinase-encoding sequence, a nucleic acid sequence encoding a peptide separation linker. Preferably, the detectable marker and the recombinase protein products are expressed by a single open reading frame.

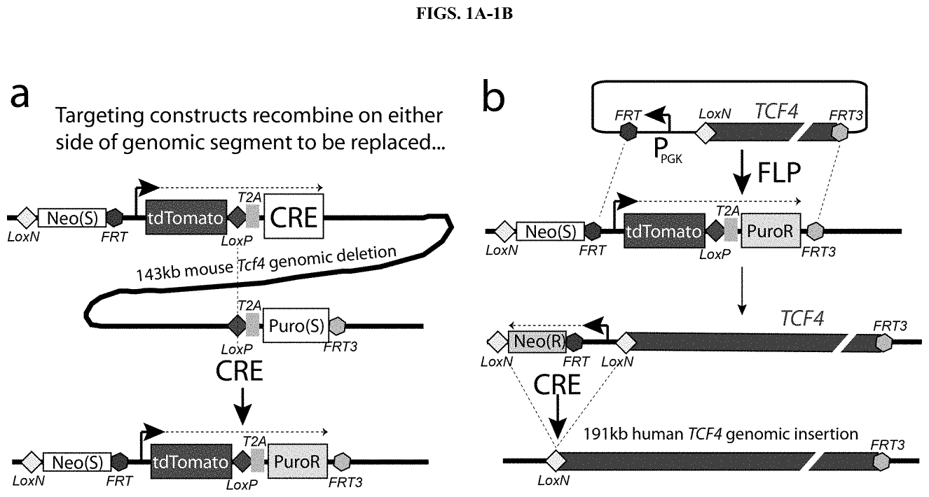

The second targeting construct can comprise a nucleic acid sequence encoding a second selectable marker flanked by a fourth recombination site and a fifth recombination site, wherein the fourth recombination site is upstream of the second selectable marker and is capable of recombining with the third recombination site in the presence of the recombinase. For example, as illustrated in FIG. 1A, the third and fourth recombination sites can be LoxP sites and the recombinase is a Cre recombinase. When the first targeting construct inserts upstream of the target endogenous gene and the second targeting construct inserts downstream of the target endogenous gene, expression of the recombinase catalyzes recombination between the third and fourth sites (e.g., LoxP sites in the illustrated embodiment of FIG. 1A), in the process excising the target endogenous gene as well as the sequence encoding the recombinase. Following excision, the resulting nucleic acid sequence inserted into the genome of the target non-human cell comprises, in order, the first recombination site, the first selectable marker, the second recombination site, the promoter, detectable marker, the third recombination site, optionally a peptide linker sequence, the second selectable marker, and the fifth recombination site. As illustrated in FIG. 1A, one embodiment of the resulting inserted construct comprises, in order, LoxN (first recombination site), Neo selection cassette, FRT (second recombination site), promoter operably linked to tdTomato (detectable marker), LoxP (third recombination site), T2A (peptide linker), Puro selection cassette, and FRT3 (fifth recombination site).

In some cases, the method further comprises selecting a non-human cell in which the endogenous non-human gene is excised by detecting expression of a detectable marker and/or selectable marker(s). The term “expression” as used herein is defined as the transcription and/or translation of a particular nucleotide sequence driven by its promoter.

To insert a human syntenic gene in place of the excised endogenous gene in the non-human cell, introduced into the non-human cell is a nucleic acid vector comprising nucleic acid sequence syntenic to the excised non-human animal endogenous gene, where the syntenic sequence is flanked on the 5′ end by a sixth recombination site and flanked on the 3′ end by a seventh recombination site. The vector further comprises a promoter operably linked to a nucleic acid sequence encoding a second recombinase and an eighth recombination site, where the fifth and seventh recombination sites recombine with each other, and where the second and eighth recombination sites recombine with each other in the presence of the second recombinase. Preferably, the first and sixth recombination sites recombine with each other in the presence of a first recombinase. As illustrated in FIGS. 1A and 1B, the first and sixth recombination sites can be LoxN sites, the second and eighth recombination sites can be FRT sites, and the fifth and seventh recombination sites can be FRT3 sites. In such cases, expression of a FLP recombinase catalyzes recombination between the FRT sites and between the FRT3 sites, resulting in insertion of the syntenic sequence in place of the excised endogenous non-human gene. In some cases, the method further comprises selecting a cell in which recombination occurred by detecting expression from a detectable marker and/or selection system.

Expression of a recombinase then catalyzes recombination between compatible recombination sites to excise nucleic acid sequences encoding the screening marker and the selection marker. In this manner, only a small portion of recombination site sequence is retained at either end of the inserted human gene. Most recombination sites have a length between 10 basepairs (bp) and 50 bp. For example, LOX and FRT sites are both 34 bp long. Some recombination sites might be longer than 50 bp, but rarely are recombination sites over 100 bp in length.

In some cases, CRISPR/Cas-catalyzed repair of double stranded breaks (DSBs) is used to promote incorporation of targeting constructs. In such cases, also introduced into the non-human cell are one or more Cas/gRNA constructs comprising one or more gRNAs having sequence complementary to at least a portion of the target endogenous non-human gene. The gRNAs expressed from the one or more Cas/gRNA constructs can associate with the endogenous target gene to which they have complementarity and generate double stranded breaks 5′ (upstream) and 3′ (downstream) to the endogenous non-human gene. The double stranded breaks will be repaired by homology-directed repair using the first and second targeting constructs, whereby the first and second targeting constructs are inserted 5′ and 3′ to the endogenous non-human target gene. In this manner, targeting constructs comprising recombination sites are placed upstream and downstream of the endogenous gene to be excised. As used herein “upstream” refers to positions 5′ of a location on a polynucleotide, and positions toward the N-terminus of a location on a polypeptide. As used herein “downstream” refers to positions 3′ of a location on nucleotide and toward the C-terminus of a location on a polypeptide. Expression of the recombinase from the first targeting construct catalyzes recombination between compatible recombination sites, whereby the endogenous non-human gene and the nucleic acid encoding the recombinase are excised and the promoter drives expression of the screening marker and selectable marker. While the examples provided herein describe the use of CRISPR/Cas systems, the methods of the instant invention may be used with other gene editing techniques. Various gene editing technologies are known to those skilled in the art and include, without limitation, homing endonucleases, zinc-finger nucleases (ZFNs), transcription activator-like effector (TALE) nucleases (TALENs), and clustered regularly interspaced short palindromic repeats (CRISPR)-CRISPR-associated protein (e.g., Cas9).

CRISPR systems belong to different classes, with different repeat patterns, sets of genes, and species ranges. A CRISPR enzyme is typically a type I or III CRISPR enzyme. The CRISPR system is derived advantageously from a type II CRISPR system. The type II CRISPR enzyme may be any Cas enzyme. The terms “Cas” and “CRISPR-associated Cas” are used interchangeably herein. The Cas enzyme can be any naturally-occurring nuclease as well as any chimeras, mutants, homologs, or orthologs. In some embodiments, one or more elements of a CRISPR system is derived from a particular organism comprising an endogenous CRISPR system, such as Streptococcus pyogenes (SP) CRISPR systems or Staphylococcus aureus (SA) CRISPR systems. The CRISPR system is a type II CRISPR system and the Cas enzyme is Cas9 or a catalytically inactive Cas9 (dCas9). Other non-limiting examples of Cas proteins include Cas 1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. A comprehensive review of the Cas protein family is presented in Haft et al. (2005) Computational Biology, PLoS Comput. Biol. 1:e60. At least 41 CRISPR-associated (Cas) gene families have been described to date.

It will be understood that the CRISPR-Cas system as described herein is non-naturally occurring in a cell, i.e. engineered or exogenous to the cell. The CRISPR-Cas system as referred to herein has been introduced in a cell. Methods for introducing the CRISPR-Cas system in a cell are known in the art, and are further described herein elsewhere. The cell comprising the CRISPR-Cas system, or having the CRISPR-Cas system introduced, according to the invention comprises or is capable of expressing the individual components of the CRISPR-Cas system to establish a functional CRISPR complex, capable of modifying (such as cleaving) a target DNA sequence. Accordingly, as referred to herein, the cell comprising the CRISPR-Cas system can be a cell comprising the individual components of the CRISPR-Cas system to establish a functional CRISPR complex, capable of modifying (such as cleaving) a target DNA sequence. Alternatively, as referred to herein, and preferably, the cell comprising the CRISPR-Cas system can be a cell comprising one or more nucleic acid molecule encoding the individual components of the CRISPR-Cas system, which can be expressed in the cell to establish a functional CRISPR complex, capable of modifying (such as cleaving) a target DNA sequence.

As used herein, the term “syntenic” refers to the conservation of order or position within two sets of chromosomes that are being compared with each other (e.g., chromosomes of two different species). Generally, syntenic regions are detected by aligning two genomes (target and reference genomes) and identifying putatively homologous gene pairs based on conservation of position in a target genome relative to a reference genome. Syntenic genes are more likely to be functional homologs.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A variety of recombinases and corresponding recombinase target sites can be used in accordance with methods provided herein. “Recombinases,” as used herein, refer to gene products and synthetic analogs thereof that catalyze recombination between a first and second polynucleotide. It is noted that recombinases typically can catalyze recombination between polynucleotide sequences in cis (i.e., on the same polynucleotide strand) or in trans (i.e., on different polynucleotide strands). As used herein, the terms “recombinase target sites” and “recombination sites” are used interchangeably and refer to polynucleotide sequences on which recombinases specifically act to induce recombination. A particular recombinase may have specificity for a single nucleic acid sequence, or a plurality of nucleic acid sequences. Such a plurality of sequences can be described by a consensus sequence. In some embodiments, a recombinase polypeptide is provided. In some embodiments, a polynucleotide encoding a recombinase polypeptide (a “recombinase polynucleotide”) is provided. Exemplary recombinases and recombinase target sites that can be used in accordance with embodiments herein include, but are not limited to, Cre-lox and FLP-FRT. The Cre-lox system, derived from bacteriophage P1, is a well-characterized recombinase and recombinase target site system (see, e.g., Lakso et al., 1992, Proc. Natl. Acad. Sci. USA 89: 6232-6236). Cre recombinase catalyzes site-specific recombination, which can excise or invert an intervening target sequence or transgene located between lox sequences. Canonically, loxP sequences are targets for Cre recombinase, but loxN or lox2272 can be used as a Cre recombinase target. Without being limited to any particular theory, Cre recombinase can work on any of the loxP or variant lox sites described herein. While Cre recombinase can induce recombination between a pair of identical lox sites (e.g., two loxP sites), Cre recombinase typically cannot induce recombination between a pair of non-identical lox sites (e.g., cannot induce recombination between a loxP and a loxN site). In some embodiments, the recombinase target sites comprise lox sites as described herein (as such, the recombinase can comprise Cre). In some embodiments, lox recombination sites are selected from, for example, loxP sites, loxN sites, or lox2272 sites as described herein.

The FLP recombinase system, derived from of Saccharomyces cerevisiae (see, e.g., O'Gorman et al., 1991, Science 251: 1351-1355; PCT publication WO 92/15694, each of which is incorporated by reference herein in its entirety) can be used to generate in vivo site-specific genetic recombination, similar to the Cre-lox system. In some embodiments, the recombinase target sites comprise FRT sites as described herein (as such, the recombinase can comprise FLPase). While FLP recombinase (FLPase) can induce recombination between a pair of identical FLP sites, it typically cannot induce recombination between a pair of non-identical FRT sites. Accordingly, in some cases, the FRTa recombination sites are FRT recombination sites, and the FRTb recombination sites are FRT3 recombination sites. In other cases, the FRTa recombination sites are FRT3 recombination sites, and the FRTb recombination sites are FRT recombination sites. In some embodiments, the first, second, and third recombinase target sites comprise FRT sites.

In some embodiments, the recombination sites are selected from lox sites for recombination catalyzed by Cre recombinase, FRT sites for recombination catalyzed by Flp recombinase (“FLPase”), Rs sites for recombination catalyzed by R recombinase, rox sites for recombination catalyzed by Dre recombinase, and gix sites for recombination catalyzed by Gin recombinase. In some embodiments, the recombination sites are attP (phage attachment) and attB (bacterial attachment) sites for recombination catalyzed by Bxb1 integrase or phiC31 integrase.

A variety of other site-specific recombinases may be employed in the methods of the present invention in place of the Cre or FLP recombinase. Alternative site-specific recombinases include, without limitation, Int recombinase of bacteriophage lambda (with or without Xis) which recognizes att sites (Weisberg et al. 1983 In: Lambda II, Hendrix et al. Eds., Cold Spring Harbor Press, Cold Spring Harbor, N.Y., pp. 211-250); the xerC and xerD recombinases of E. coli which together form a recombinase that recognizes the 28 bp dif site (Leslie and Sherratt 1995 EMBO J 14:1561); the Int protein from the conjugative transposon Tn916 (Lu and Churchward 1994 EMBO J 13:1541); TpnI and the β-lactamase transposons (Levesque 1990 J Bacteriol 172:3745); and Tn3 resolvase (Flanagan et al. 1989 J Mol Biol 206:295 and Stark et al. 1989 Cell 58:779).

Any appropriate nucleic acid vector can be used with the methods provided herein. In some cases, the nucleic acid vector is a bacterial artificial chromosome (BAC). As used herein, the term “bacterial artificial chromosome (BAC)” refers to plasmid expression vectors that can stably hold several hundred kilobases (kb) of foreign (i.e., exogenous) DNA and can either integrate with the genome of a mammalian cell or be lost upon mammalian host cell replication. BAC constructs comprise promoters and, optionally, other sequences. The term “expression vector” or “vector” as used herein refers to a recombinant DNA molecule containing a desired coding sequence and appropriate nucleic acid sequences necessary for the expression (i.e., transcription and/or translation) of the operably linked coding sequence in a particular host organism. Other appropriate nucleic acid vectors include, without limitation, a yeast artificial chromosome, bacterial plasmid, phagemid, shuttle vector, cosmid, virus, chromosome, mitochondrial DNA, plastid DNA, and nucleic acid fragment.

In some embodiments, the targeting construct (e.g., first or second targeting construct) includes a 2A element, which is a nucleic acid sequence encoding a 2A peptide. 2A peptides, first discovered in picornaviruses, are short (about 18-22 amino acids) and produce equimolar levels of multiple genes from the same mRNA. Sequences encoding commonly used 2A peptides such as T2A (thosea asigna virus 2A), P2A (porcine teschovirus-1 2A), E2A (equine rhinitis A virus), and F2A (foot and mouth disease virus 2A), are known and available to practitioners in the art. For example, peptide T2A (thosea asigna virus 2A) is encoded by amino acid sequence EGRGSLLTCGDVEENPGP (SEQ ID NO:1). In some cases, one or more glycine-serine-glycine spacers (GSG) is added to the 5′ end of the peptide to improve cleavage efficiency.

Various promoters can be operably linked with a nucleic acid comprising the coding region of the gene product of interest in the vectors to drive expression of the gene product of interest in accordance with embodiments herein. Examples of promoters, include, but are not limited to, viral promoters, plant promoters, and mammalian promoters. Examples of viral promoters include, but are not limited to cytomegalovirus (CMV) immediate early promoter, CAG promoter (which is a combination of the CMV early enhancer element and chicken beta-actin promoter, described in Alexopoulou et al. BMC Cell Biology 9:2, (2008)), simian virus 40 (SV40) promoter, the 35S RNA and 19S RNA promoters of cauliflower mosaic virus (CaMV) described in Brisson et al., Nature 1984, 310:511-514, the coat protein promoter to tobacco mosaic virus (TMV), and any variants thereof. Examples of mammalian promoters include, but are not limited to, human elongation factor 1α-subunit (EF1-1α) promoter, human ubiquitin C (UCB) promoter, murine phosphoglycerate kinase-1 (PGK) promoter, and any variants thereof. As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter is capable of affecting the expression of that coding sequence (i.e., the coding sequence is under the transcriptional control of the promoter).

In some embodiments, the promoter is operably linked to a polynucleotide encoding one or more polypeptides of interest. To increase expression from the promoter in vectors in accordance with embodiments herein, the promoter can comprise a transcriptional enhancer.

In some cases, a targeting construct (e.g., a first or second targeting construct) comprises a screening marker. As used herein, the term “screening marker” or “reporter gene” refers to a gene encoding a protein that may be assayed (screened) directly or indirectly. In a particular embodiment, the reporter can be directly assayed. Examples of reporter genes include, but are not limited to, bioluminescence catalyzing enzymes (e.g. luciferase), fluorescent protein (e.g., RFP (e.g., monomer red fluorescent protein), GFP (e.g., turboGFP (Evrogen; Russia))), chloramphenicol acetyltransferase, β-galactosidase, alkaline phosphatase, and horseradish peroxidase.

In some cases, a targeting construct (e.g., a first or second targeting construct) comprises a selectable marker. As used herein, the term “selectable marker” or “marker gene” refers to a gene which encodes an enzyme having an activity that confers resistance to an antibiotic or drug upon the cell in which the selectable marker is expressed. Examples of selectable markers include, without limitation, hygromycin phosphotransferase, blasticidin-S-deaminase (BSD), puromycin acetyltransferase (PAT), and neomycin phosphotransferase II (NPTII).

Any appropriate method of introducing nucleic acid sequences or constructs can be used for the recombination and gene editing methods described herein. In some cases, nucleic acids are transfected into a non-human host cell. The term “transfected” or “transformed” or “transduced” as used herein refers to a process by which exogenous nucleic acid is transferred or introduced into the host cell (e.g., mouse cell). A “transfected” or “transformed” or “transduced” cell is one which has been transfected, transformed or transduced with exogenous nucleic acid. The cell includes the primary subject cell and its progeny.

In another aspect, provided herein is a genetically modified non-human cell comprising in its genome a human gene in place of an endogenous syntenic gene. Also provided herein are genetically modified non-human animals obtained from such genetically modified cells. Preferably, the animal is obtained from a genetically modified cell produced according to the methods of this disclosure. Because the genetically modified cell or animal comprises a human gene in place of an endogenous gene, the animal or cell is considered to be “transgenic.” As used herein, the term “transgenic” refers to a cell and/or animal having a genome into which genetic material from a different organism has been artificially introduced. Genetic material that is artificially introduced or is about to be artificially introduced into the genome of a cell and/or animal is referred to herein as a “transgene” or “transgenic DNA sequence.” The transgenic DNA sequences are integrated in all or a portion of the animals cells, especially in the germ cells. The integration into the genome may be transient or stable. Preferably, the integration is stable.

The genetically modified cells and animals of this disclosure are non-human and include, without limitation, rodents (such as mice, rats, guinea pigs), farm animals (such as pigs, goats, sheep, cows, horses), poultry (e.g., avian species such as chickens, ducks, geese, turkeys, etc.), lagomorphs (e.g., rabbits), and domestic animals (such as dogs and cats).

In some cases, the non-human animal cell is an embryonic stem (ES) cell, stem cell, somatic cell, or induced pluripotent stem cell (iPS cell). Upon genetic modification, the modified cells can be expanded in vitro. In some cases, a nucleus from a genetically modified cell is transferred to an enucleated oocyte or one-cell embryo in a process referred to as reproductive cloning. The reconstructed embryos are then implanted into surrogate recipients. These genetically modified embryos retain the ability to support fetal development. A change in the genomic sequence of the embryos will be passed on to all other cells derived directly from the modified embryos including the germ line. The resulting offspring are heterozygous and homozygous for the human gene replacement.

In some cases, the non-human animal cell is a mouse ES (embryonic stem) cell and the gene replacement steps provided herein are performed by introducing the constructs into a mouse ES cell. To generate mice from these modified ES cells, the modified ES cells are injected into a host blastocyst, and the chimeric blastocyst is implanted into a surrogate mother. The chimeric mice that are born are then bred to ensure germline transmission of the modified genomic locus. In another aspect, the gene replacement approach provided herein can be used to modify single cell-stage embryos of non-human animals. Genetically modified non-human animals would be generated from these modified embryos by implanting them into surrogate mothers.

As described in the Examples that follow, the gene replacement methods provided herein permit one to generate a precisely matched set of mouse lines comprising syntenic human sequence in less than five months, from first ES cell transfection to weaned chimeric mice.

Depending on the genetic modification, it may be advantageous to analyze a modified non-human animal genetically, molecularly, and/or behaviorally. It may be particularly advantageous to analyze the modified non-human animal for suitability as a model of human disease. For example, in some cases, a genetically modified cell (e.g., a modified non-human cell) is genetically edited to introduce a human gene for which one or more particular mutations (e.g., single nucleotide polymorphisms) are associated with a human disease or disorder. As described in Example 1, approximately 4% of the Caucasian population in the United States may eventually lose their vision to Fuchs endothelial corneal dystrophy (FECD), a progressive, degenerative disease of the eye characterized by dysfunction of the corneal epithelium. FECD is the result of a pathogenic CTG-repeat expansion mutation present in a non-conserved segment of the third intron of the longest primary transcript of the human Transcription Factor 4 (TCF4) gene. However, classic approaches for generating animal models are not adequate to generate an experimental system that will let us directly measure the molecular impacts of these pathogenic CTG-repeat expansions on the human gene in which it occurs. As demonstrated in Example 1, the methods provided herein are useful to produce genetically modified animals comprising the human TCF4 gene in place of the endogenous non-human gene. Such genetically modified animals provide an improved model for studying pathology of human disease in an animal model. They also provide a unique screening tool to identify candidate therapeutic agents.

As another example, the human microtubule-associated protein tau (MAPT) gene encodes tau, the predominant component of neurofibrillary tangles that are neuropathological hallmarks of Alzheimer's disease (AD) and other neurodegenerative disorders. Genetic variation in the MAPT gene is associated with AD risk, but the precise roles of particular variants of the H1 and H2 alleles of the human MAPT gene are unclear. As described in the Example 2, the inventors developed a novel mouse line in which the entire Mapt coding and regulatory region (157 kb) have been completely and precisely replaced by the corresponding sequences in the human genome (190 kb). The MAPT gene replacement mouse line can be used to fully investigate the molecular impacts of the cumulative sequence differences between the H1 and H2 alleles. In particular, the methods described herein make it possible to produce precisely matched lines of mice that differ in their genomic sequences at only those non-coding variant sites within the H1 and H2 human MAPT sequences.

The advantages of the gene replacement (GR) technology described herein are multifold and include, for example, the development of GR mouse or other non-human animal lines in which an entire human gene precisely replaces the whole endogenous gene. The presence of a single copy of the entire human syntenic gene in the defined, endogenous locus will permit us to assay the biological activity of non-coding variants in the human gene that are absent in the related mouse gene. No other similarly modified animals exist. Consequently, it has not been possible to determine the in vivo effects of genetic variations that differentiate particular alleles of a gene of interest. The second innovative aspect is the examination of human haplotypes in a living brain. For example, studies in cultured cells and post-mortem brain show differences in the expression and splicing of tau in human MAPT haplotypes. As described in Example 2, MAPT GR mice are particularly useful to validate and extend these results. In particular, the use of MAPT GR mice makes it possible to evaluate and compare the dynamic progression of molecular endophenotypes of tau in young and old subjects, which is the third innovative aspect of this technology. Tauopathies develop in the context of an aging brain, which is characterized by region-specific changes in volume and neural activity. It is reasonable to expect that the spatial, temporal or splicing patterns of tau mRNA expression in subjects with different MAPT haplotypes will differ, but it is harder to predict their effects on protein expression. Our studies will not only evaluate the extent to which aging influences MAPT haplotype-associated changes in transcription but also the effects of aging on translation and post-translational modifications of tau. Using MAPT GR mice permit assessing molecular endophenotypes in different brain regions, which highlights the fourth innovative aspect of this technology. Recently, the development of iPSC technology has advanced our understanding of the effects of genome-wide association studies (GWAS) hits on the biochemistry and cell biology of differentiated human neurons. MAPT expression, however, is differentially regulated by brain region and cell type. The use of MAPT GR mice will allow us to study the degree to which different brain regions modulate the effects of MAPT haplotypes.

As another example of the advantages of the gene replacement (GR) technology described herein, the methods of this disclosure can be performed to obtain to mice in which the mouse gene encoding amyloid precursor protein (App) with a syntenic human genomic segment. APP is the precursor molecule that is cleaved to generate beta amyloid peptides. Beta amyloid is a component of amyloid plaques associated with Alzheimer's disease (AD). AD is the most common neurodegenerative disease affecting 60-80% of all dementia cases. There is no cure for AD, as such the study of this disease is an active area of basic and translational research. There are several animal models of AD, each with its distinct advantages and disadvantages. As described in Example 4, the gene replacement methods of this disclosure have been used to produce an APP-gene replacement (GR) mouse comprising the human APP gene. In some cases, mice comprising the full-length human APP gene can be further genetically modified to introduce clinically relevant mutations associated with various disease phenotypes. In such cases, the APP-GR mouse can provide a valuable tool for basic and clinical trial research since it allows the genetic modification of the human transgene to achieve a particular phenotype.

Precisely matched sets of animal models developed using the methods provided herein will allow researchers to evaluate the molecular impact of pathogenic mutations within the context of the human genomic sequence in which they occur in patients, and these mouse lines can contain all of the potential human therapeutic targets ranging from the full genomic DNA sequences to all of the RNA transcription variants and protein products that they encode. Furthermore, because the genomic sequences of these matched sets will differ only at sequences specifically changed in each line, any significant molecular differences between these lines can be confidently attributed to the pathogenic mutation in the experimental lines, and any therapeutic agents found to effectively correct these dysfunctions could be expected to have direct therapeutic value to patients.

In some cases, genetically modified non-human animals of the invention are useful in drug discovery and development including screening for potential therapeutic agents. In some cases, the methods comprise contacting or administering a candidate test agent to a genetically modified non-human animal, where the modified animal comprises a human syntenic gene in place of an endogenous gene and the human syntenic gene is associated with a particular disease or disorder. The methods further comprise, following contacting or administration of the test agent, analyzing the genetically modified animal for a molecular, physiological, and/or behavioral change relative to a similarly modified animal which has not received the test agent. Exemplary test agents include, without limitation, small molecules, proteins, peptides, antibodies, oligonucleotides, small interfering RNAs (siRNAs), polynucleotides, peptidomimetics, cytotoxic agents, pharmaceutical agents, and infectious agents.

In some cases, analyzing comprises detecting at least one positive or negative effect of the test agent on morphology or life span of a genetically modified non-human animal or cells and tissues thereof. In some cases, detecting comprises performing a method selected from the group consisting of RNA sequencing, gene expression profiling, transcriptome analysis, cell proliferation assays, metabolome analysis, detecting reporter or sensor, protein expression profiling, Forster resonance energy transfer (FRET), metabolic profiling, and microdialysis. In some cases, the agent can be screened for an effect on gene expression, and detecting can comprise assaying for differential gene expression relative to a control non-human animal or tissue or cells derived therefrom.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

While the present invention has been described in some detail for purposes of clarity and understanding, one skilled in the art will appreciate that various changes in form and detail can be made without departing from the true scope of the invention.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. This applies regardless of the breadth of the range.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

The foregoing description and Examples detail certain embodiments. It will be appreciated, however, that no matter how detailed the foregoing may appear in text, the invention may be practiced in many ways and the invention should be construed in accordance with the appended claims and any equivalents thereof.

### EXAMPLES

### Example 1—Two-Step Process for Replacing a Gene of Interest in an Animal Genome with the Orthologous Human Gene

The longest primary transcript of the human Transcription Factor 4 (TCF4) gene is 414153 bp long, but the open-reading frame in the final processed mRNA is only 2322 bp (0.56% of the primary transcript). Approximately 4% of the Caucasian population in the US may develop Fuchs endothelial corneal dystrophy (FECD), which is a degenerative disease of the eye associated with a pathogenic CTG-repeat expansion mutation present in a non-conserved segment of the third intron of the longest primary TCF4 transcript³. However, it cannot be predicted what impact this pathogenic mutation might be having on this transcript (or on any of the other 20 TCF4 transcripts that originate at positions 5′ or 3′ to this mutation⁴). Classic approaches for generating animal models are not adequate to generate an experimental system that will let us directly measure the molecular impacts of this mutation on the human gene in which it occurs.

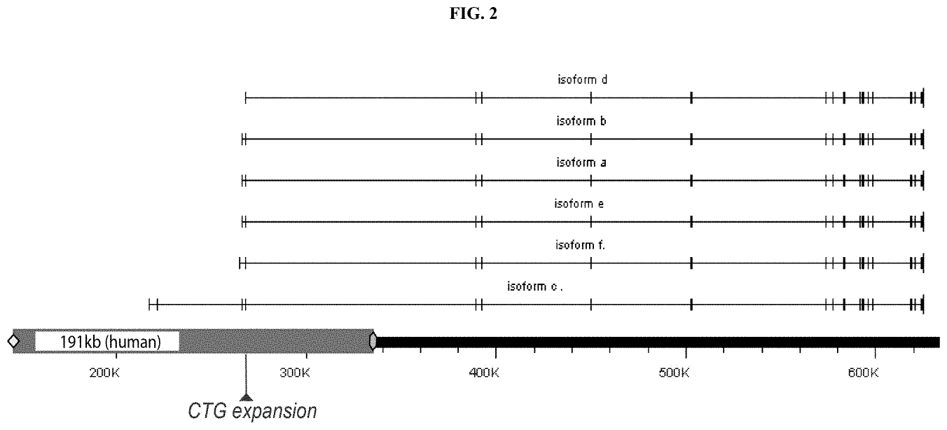

This example describes a two-step process for replacing a gene of interest in an animal genome with the orthologous human gene: 1) deletion of the gene in the target animal genome, and 2) insertion of the syntenic sequence from the human genome. As illustrated in FIG. 1A, we first generated a mouse embryonic stem (ES) cell line in which a 143 kb portion of the mouse Tcf4 gene is deleted and replaced by a site-specific recombination target cassette. Shown in FIG. 1B, we then integrated a set of human TCF4 gene variants (191 kb fragments) into this prepared ES cell line, either without (wt control) or with (experimental) the CTG expansion mutation that causes Fuchs endothelial corneal dystrophy (FECD). (FIG. 2). As described below, these proof-of-concept experiments demonstrate that the two-step process can be used generate a precisely matched set of mouse lines in less than five months, from the first ES cell transfection to weaned chimeric mice.

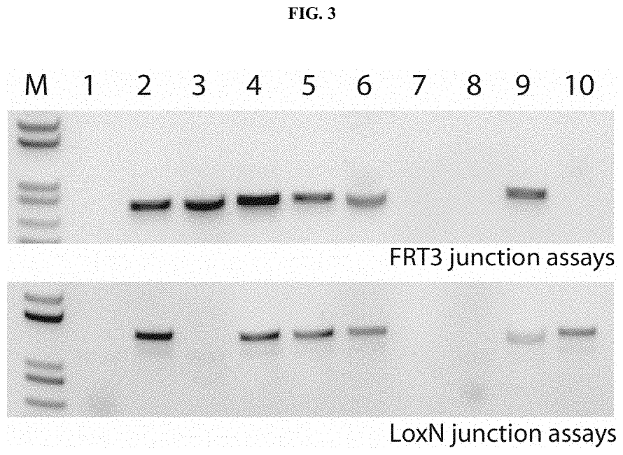

A single ES cell transfection experiment was performed. Unexpectedly, our single transfection reaction resulted in a series of molecular events occurred: 1) two different plasmid constructs recombined on either end of the region of the Tcf4 gene to be replaced, and 2) the Tcf4 genomic segment between these two constructs was deleted by a Cre-mediated reaction. Importantly, we incorporated a powerful selection scheme into the design of this reaction, which allowed us to screen a total of only 20 ES clones and identify 5 clones that had been correctly modified (FIG. 3). The total duration of this experiment, from transfection to identification of the correct clones, was 18 days.

Replacing a 143 kb Portion of Tcf4 in Mouse ES Cells with an FRT “Flp-In” Selection Cassette

Using CRISPR endonuclease cleavage, we dramatically enhanced the efficiency of the homologous-recombination reactions in which these constructs were correctly targeted to the flanking insertion target sites. In practice, this involved co-transfecting a separate Cas9/sgRNA plasmid with each of the two targeting constructs, for a total of four different plasmids used in each transfection of the C57BL/6N ES cell line (JAX; PRX-B6N #1). Because we did not validate the efficiency of the predicted CRISPR cleavage reagents prior to these experiments, we actually designed and constructed two alternative Cas9/sgRNA plasmids⁵ designed to cleave either of two overlapping target sequences (A1: GTGAAGACATCCCCCACTGT (SEQ ID NO:2) or A2: TGTGAAGACATCCCCCACTG (SEQ ID NO:3); B1: GGCAAGATGAGATATTTATG (SEQ ID NO:4) or B2: AAGATGAGATAT-TTATGTGG (SEQ ID NO:5)). Experimentally, we then performed four different transfection reactions, each with alternative combinations of these Cas9/sgRNA plasmids: A1/B1, A1/B2, A2/B1, or A2/B2. Utilizing CRISPR technology also allowed us to use targeting constructs with fairly short homology arms (specifically either 445 bp and 539 bp, or 462 bp and 473 bp), which in turn allowed us to screen the colonies with simple PCR reactions (FIG. 3).

Incorporating the Cre-mediated deletion event is a critical factor in the success of this approach. Our repeated, unsuccessful alternative experiments in which we have tried to identify CRISPR-mediated deletion events of more than a few kb in length has led to our firm conviction that the homologous-recombination repair mechanisms that drive these reactions strongly disfavor the loss of significant segments of genomic sequences. Once our targeting constructs were correctly incorporated on either side of the Tcf4 gene fragment, however, our data suggests that the Cre recombinase readily catalyzed recombination between the flanking loxP sites and efficiently removed the intervening 143 kb fragment. The targeting construct on one end of the Tcf4 region consisted in part of a cassette that expresses tdTomato and Cre recombinase, separated by a loxP recombination site and a T2A peptide separation linker⁶. The other flanking construct consisted of a promoter-less Puromycin resistance gene fused in-frame with a loxP/T2A linker. By subjecting the transfected ES cell culture to Puromycin selection and picking only colonies that fluoresced red, we were able to highly enrich for those cells that had undergone the desired series of recombination events (5 correct out of 20 clones assayed, FIG. 3). The final ES cell product of these recombination reactions contains a cassette designed to allow us to select for Flp-mediated incorporation of the human TCF4 genomic fragment at the ΔTcf4 locus (FIGS. 1A-1B).

Modifying a 191 kb Fragment of the Human TCF4 Gene and Integration into the ΔTcf4 ES Cell Target Locus

We modified the BAC clone RP11-230M3 by introducing 1) a loxN-PGK (mouse phosphoglycerate kinase 1) promoter-FRT cassette at one end of the TCF4 genomic sequence, and 2) an FRT3 recombination site variant⁷ at the other end, while simultaneously trimming the unwanted sequences from the ends of the genomic fragment. We then introduced an expanded CTG-repeat tract (replicating the 83xCTG repeat mutation) into this modified BAC, and completely sequenced both the control (wt) and experimental (83xCTG repeat mutation) BAC constructs (Illumina, University of Minnesota Genomics Center, 190538 bp in the wt clone flanked by FRT3 and loxN). We co-transfected this set of BAC clones together with a FLP-expression plasmid (pCAGGS-FLPe; Gene Bridges) into the modified ΔTcf4 ES cell line (above) and selected for G418-resistant colonies. Although we only obtained a modest number of clones from this experiment (two wt and three 83xCTG TCF4 lines), subsequent analyses demonstrated that all of these clones carry the full TCF4 fragment correctly inserted at the expected location. We have now generated mouse lines from both the wt and 83xCTG ES cells, and will eliminate the loxN-flanked selection cassette from these lines by mating to Sox2-Cre (JAX) female mice on a C57BL/6 background (“Cre” reaction in FIG. 1B).

We generated precisely engineered BAC constructs using an efficient, markerless “recombineering” approach⁸ that we developed and routinely use in our lab, and can use this approach to make essentially any desired modification to these clones (insertions, deletions, point mutations, etc.). We have to date successfully generated more than a dozen “Flp-In” BAC mouse lines using procedures essentially identical to the second step of our Gene-Replacement approach. For these earlier projects, however, we integrated genomic BAC constructs in single-copy at the intergenic Col1a1 target⁹, a “neutral” integration site that we know to be favorable for expression and that does not disrupt any normal gene functions. We have routinely found that essentially all of the Hygro-resistant ES clones obtained by the Col1a1 Flp-in procedure have a BAC correctly integrated at the target FRT site. This high specificity is achieved because the promoter-less hygromycin resistance gene at the target locus is only expressed when the promoter and ATG translation initiation sequence on the BAC construct begins to drive expression following site-specific recombination between the FRT sequences in the BAC and in the genome. Because we do not wish to retain the Pgk-FRT-HygoR cassette at the integration locus in Gene-Replacement projects, we designed those Flp recombination events to generate a selection cassette flanked by loxN sites' that can be removed with a subsequent Cre recombination excision event (see FIG. 1B). The mouse lines generated using this approach therefore only retain a 34 bp loxN sequence at one end of the inserted 191 kb human TCF4 genomic sequence and a 34 bp FRT3 sequence at the other.

### REFERENCES

1 Moraes, F. & Goes, A. A decade of human genome project conclusion: Scientific diffusion about our genome knowledge. Biochem Mol Biol Educ 44, 215-223 (2016).
2 Dapas, M., Kandpal, M., Bi, Y. & Davuluri, R. V. Comparative evaluation of isoform-level gene expression estimation algorithms for RNA-seq and exon-array platforms. Brief Bioinform 18, 260-269 (2017).
3 Wieben, E. D. et al. A common trinucleotide repeat expansion within the transcription factor 4 (TCF4, E2-2) gene predicts Fuchs corneal dystrophy. PLoS One 7, e49083 (2012).
4 Sepp, M., Kannike, K., Eesmaa, A., Urb, M. & Timmusk, T. Functional diversity of human basic helix-loop-helix transcription factor TCF4 isoforms generated by alternative 5′ exon usage and splicing. PLoS One 6, e22138 (2011).
5 Ran, F. A. et al. Genome engineering using the CRISPR-Cas9 system. Nat Protoc 8, 2281-2308 (2013).
6 Szymczak, A. L. et al. Correction of multi-gene deficiency in vivo using a single ‘self-cleaving’ 2A peptide-based retroviral vector. Nat Biotechnol 22, 589-594 (2004).
7 Turan, S., Kuehle, J., Schambach, A., Baum, C. & Bode, J. Multiplexing RMCE: versatile extensions of the Flp-recombinase-mediated cassette-exchange technology. J. Mol. Biol. 402, 52-69 (2010).
8 Benzow, K. A. & Koob, M. D. Markerless modification of trinucleotide repeat loci in BACs. Methods Mol Biol 1010, 265-276 (2013).
9 Beard, C., Hochedlinger, K., Plath, K., Wutz, A. & Jaenisch, R. Efficient method to generate single-copy transgenic mice by site-specific integration in embryonic stem cells. Genesis 44, 23-28 (2006).
10 Livet, J. et al. Transgenic strategies for combinatorial expression of fluorescent proteins in the nervous system. Nature 450, 56-62 (2007).
11 Feil, R., Wagner, J., Metzger, D. & Chambon, P. Regulation of Cre recombinase activity by mutated estrogen receptor ligand-binding domains. Biochem. Biophys. Res. Commun. 237, 752-757, (1997).
12 Koob, M. & Szybalski, W. Cleaving yeast and Escherichia coli genomes at a single site. Science 250, 271-273 (1990).
13 Koob, M. & Szybalski, W. Preparing and using agarose microbeads. Methods Enzymol 216, 13-20 (1992).
14 Boussif, O. et al. A versatile vector for gene and oligonucleotide transfer into cells in culture and in vivo: polyethylenimine. Proc. Natl. Acad. Sci. USA 92, 7297-7301 (1995).
15 Yang, H. et al. One-step generation of mice carrying reporter and conditional alleles by CRISPR/Cas-mediated genome engineering. Cell 154, 1370-1379, (2013).

### Example 2—Complete Replacement of the Mouse Mapt Gene Locus with Human MAPT

Although more than 400 Late Onset Alzheimer's Disease (LOAD) clinical trials have been performed in the past 15 years, the failure rate has been >99%². These failures have given rise to the view that effective treatment will require shifting the timing of interventions to early stages of disease, before widespread neurodegeneration occurs. Therefore, there is a critical need for identifying the molecular processes that predispose individuals to LOAD and related disorders. Recent advances in identifying genes that alter disease risk have the potential for providing such knowledge. Ascertaining the pathological endophenotypes of these genetic risk factors could accelerate the development of therapies that target key early molecular processes.

One well-established genetic risk factor is MAPT. There are two major alleles of Chr17q21.31, termed H1 and H2. The H2 haplotype arose through the inversion of a ˜900 Kb segment of Chr17q21.31 which spans several genes, including MAPT. Many genome-wide association studies (GWAS) show a protective effect of the H2 allele. The highest H1:H2 odds ratios (OR) have been reported in the primary tauopathies PSP and corticobasal degeneration (CBD). In PSP, the H1:H2 OR is as high as 5.5³. In CBD, OR in the range of 2.2-3.5 have been reported^{4,5}. Lower H1:H2 OR have been found in secondary tauopathies. In Parkinson's disease (PD), the H1:H2 OR ranges from 1.3-1.9^{6,7}. When only PD patients with dementia were studied, the OR increased to 3.7⁷, suggesting that tau dysregulation may disproportionately promote cognitive symptoms. In LOAD, significant H1:H2 OR ranging from 1.1-1.6 were found in non-APOE4, but not APOE4, subjects^{8,9}. The relatively low OR in LOAD may reflect a “two-hit” mechanism at work, a hypothesis that is supported by the observation that an association between MAPT haplotypes and CSF tau occurred only in subjects with low CSF Aβ42 levels¹⁰.

Most geneticists agree that the ˜900 Kb inversion per se does not confer the protective effects of H2. Although there is no agreement about exactly which genetic determinants are involved, many researchers believe that risk is mediated by allelic variants in H1 and H2 MAPT that segregated due to the inability of the H1 and H2 alleles to recombine. In post-mortem brain of LOAD subjects, H2 correlated with reduced MAPT gene expression¹¹. In contrast, in post-mortem brain of control subjects, H2 did not associate with lower MAPT gene expression, but instead with reduced 4R:3R tau transcripts¹². One possible explanation for the reported inconsistencies is that the effects of MAPT variants are dynamic, changing with age and disease stage, which are difficult to evaluate in static studies of post-mortem tissue.

Generating GR1 (H1) and GR2(H2) Mice.

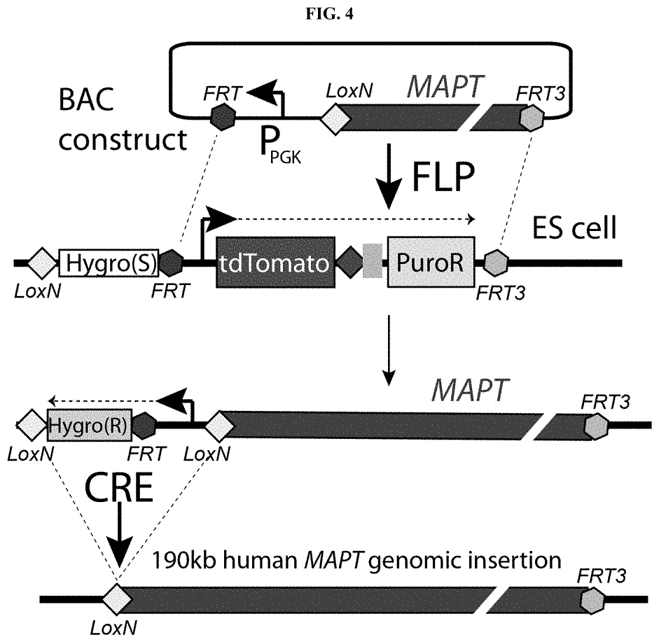

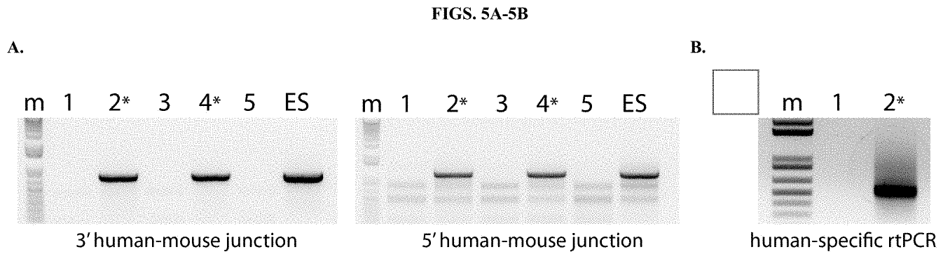

Replacing the mouse Mapt gene with the human MAPT gene is a two-step process: 1) deletion of the mouse Mapt gene in the genome of a mouse ES cell line, and 2) insertion of the syntenic sequence from the human genome. We have achieved these goals by a) generating an ES cell line in which a 157 kb portion of the mouse genome encompassing the Mapt gene is deleted and replaced by a site-specific recombination target cassette, and b) integrating a human MAPT gene construct (190 kb fragment) into this prepared ES cell line (FIGS. 5A-5B). We deleted the Mapt gene in a C57BL/6N ES cell line (JAX; PRX-B6N #1) with a single CRISPR recombination procedure in which two different plasmid constructs recombined on either end of the region of the Mapt gene to be replaced, and the intervening genomic segment was deleted by a Cre-mediated reaction. The final ES cell product of these recombination reactions contains a cassette that allows us to select for Flp-mediated incorporation of the human MAPT genomic fragment at the ΔMapt locus (FIG. 4). Importantly, all mouse lines generated in this MAPT GR series will be generated using this same ΔMapt ES cell clone.

We generate precisely engineered BAC constructs using an efficient, markerless “recombineering” approach²³ that we developed and routinely use to make essentially any desired modification to these clones (insertions, deletions, point mutations, inter-BAC recombination, etc.). We have to date successfully generated more than a dozen “Flp-In” BAC mouse lines using procedures essentially identical to the second step of our Gene-Replacement approach. For these earlier projects, however, we integrated genomic BAC constructs in single-copy at the intergenic Col1a1 target²⁴, a “neutral” integration site that we know to be favorable for expression and that does not disrupt any normal gene functions. We have routinely found that essentially all of the Hygro-resistant ES clones obtained by the Col1a1 Flp-in procedure have a BAC correctly integrated at the target FRT site. This high specificity is achieved because the promoter-less hygromycin resistance gene at the target locus is only expressed when the promoter and ATG translation initiation sequence on the BAC construct begins to drive expression following site-specific recombination between the FRT sequences in the BAC and in the genome.

Because we do not wish to retain the Pgk-FRT-HygoR cassette at the integration locus in Gene-Replacement projects, we designed Flp recombination events to generate a selection cassette flanked by loxN sites²⁵ that can be removed with a subsequent Cre recombination excision event (see FIG. 4). Therefore, mouse lines generated using this approach retain only a 34 bp loxN sequence at one end of the inserted 190 kb human MAPT genomic sequence and a 34 bp FRT3 sequence at the other.

Discussion

We generated a novel mouse line in which the entire Mapt coding and regulatory region (157 kb) have been completely and precisely replaced by the corresponding sequences in the human genome (190 kb). To generate these mice we generated a modified BAC construct using clones from the human RPCI-11 BAC library¹⁵. Subsequent sequence analysis of our completed construct drew our attention to the fact that the anonymous DNA donor from whom this library is derived had one H1 and one H2 MAPT allele. Because many of the clones from this library have been completely sequenced as part of the Human Genome Project, we have been able to assemble the entire H1 and H2 haplotype sequences from this individual, and have each of these alleles readily available in BAC clones. We are now in a unique position to fully investigate the molecular impacts of the cumulative sequence differences between H1 and H2, given 1) our knowledge of the complete H1 and H2 allele sequences from this anonymous donor, and 2) the availability of fully sequenced H1 and H2 overlapping BAC clones (669E14 & 105N13 and 769P22 & 80L9, respectively); together with our ability to 3) integrate these MAPT alleles in place of the Mapt gene in the mouse genome in the same ES cell clone and thereby 4) generate precisely matched lines of mice that differ in their genomic sequences at only those non-coding variant sites within the H1 and H2 human MAPT sequences.

Although superficially similar, the MAPT GR mice generated and described here are very different from any existing mouse line in which a BAC or PAC human genomic clone has been randomly integrated into the mouse genome. To illustrate these differences, we will use as an example for comparison the MAPT PAC “htau” transgenic line generated by Duff and Davies' by pronuclear injection of a circular PAC MAPT clone (H1 haplotype). The uncontrolled and uncharacterized transgene variables in this model include 1) the point(s) at which this circular PAC DNA was broken as it recombined into the genome; 2) the integration site and sequences within the mouse genome disrupted by this integration event; 3) the copy number (estimated three and five copies); and 4) the configuration of the PAC and/or PAC fragments within the transgene array. In their original manuscript the Duff and Davies group describe this MAPT PAC transgene as being “functionally intact”, in that alternatively spliced MAPT transcripts are present and human tau is over-expressed at levels higher than the mouse tau. However, this not a reliable indicator of the integrity of large DNA transgenes, given that a full genome sequence analysis of a similarly generated mouse line originally thought to contain a “functionally intact”, potentially single-copy BAC transgene revealed that in fact the highly rearranged transgene consisted of a random array of 6 partial fragments of the original BAC²². Any of these uncontrolled transgene variables can have profound and unpredictable impacts on the regulation and expression of the MAPT gene encoded within the original human genomic fragment, and, most importantly for the purposes of this project, none can be controlled or be reproduced, and would hopelessly confound analyses of any similar MAPT (H2 or H1) model we might make. In sharp contrast, the genomes of all subsequent models generated in the MAPT GR series of mouse lines will precisely match the initial MAPT (H2) GR line except for those MAPT nucleotide differences that we specifically introduce. As a result, all of the molecular endophenotype differences between the precisely matched GR lines can be reliably and reproducibly attributed to these sequence variants. Furthermore, by fully replacing the endogenous Mapt gene with its human counterpart, a) the MAPT gene in these lines will be situated in the only location within the mouse genome that has specifically evolved to express the tau gene, and b) the MAPT GR lines will not express any endogenous mouse tau to confound our analyses.

### REFERENCES

1 Pennisi, E. Genetics. 17q21.31: not your average genomic address. Science 322, 842-845 (2008).
2 Cummings, J. L., Morstorf, T. & Zhong, K. Alzheimer's disease drug-development pipeline: few candidates, frequent failures. Alzheimers Res Ther 6, 37 (2014).
3 Hoglinger, G. U., Melhem, N. M., Dickson, D. W., Sleiman, P. M., Wang, L. S., Klei, L., Rademakers, R., de Silva, R., Litvan, I., Riley, D. E., van Swieten, J. C., Heutink, P., Wszolek, Z. K., Uitti, R. J., Vandrovcova, J., Hurtig, H. I., Gross, R. G., Maetzler, W., Goldwurm, S., Tolosa, E., Borroni, B., Pastor, P., Group, P. S. P. G. S., Cantwell, L. B., Han, M. R., Dillman, A., van der Brug, M. P., Gibbs, J. R., Cookson, M. R., Hernandez, D. G., Singleton, A. B., Farrer, M. J., Yu, C. E., Golbe, L. I., Revesz, T., Hardy, J., Lees, A. J., Devlin, B., Hakonarson, H., Muller, U. & Schellenberg, G. D. Identification of common variants influencing risk of the tauopathy progressive supranuclear palsy. Nat Genet 43, 699-705 (2011).
4 Houlden, H., Baker, M., Morris, H. R., MacDonald, N., Pickering-Brown, S., Adamson, J., Lees, A. J., Rossor, M. N., Quinn, N. P., Kertesz, A., Khan, M. N., Hardy, J., Lantos, P. L., St George-Hyslop, P., Munoz, D. G., Mann, D., Lang, A. E., Bergeron, C., Bigio, E. H., Litvan, I., Bhatia, K. P., Dickson, D., Wood, N. W. & Hutton, M. Corticobasal degeneration and progressive supranuclear palsy share a common tau haplotype. Neurology 56, 1702-1706 (2001).
5 Pittman, A. M., Myers, A. J., Abou-Sleiman, P., Fung, H. C., Kaleem, M., Marlowe, L., Duckworth, J., Leung, D., Williams, D., Kilford, L., Thomas, N., Morris, C. M., Dickson, D., Wood, N. W., Hardy, J., Lees, A. J. & de Silva, R. Linkage disequilibrium fine mapping and haplotype association analysis of the tau gene in progressive supranuclear palsy and corticobasal degeneration. J Med Genet 42, 837-846 (2005).
6 Consortium, U. K. P. s. D., Wellcome Trust Case Control, C., Spencer, C. C., Plagnol, V., Strange, A., Gardner, M., Paisan-Ruiz, C., Band, G., Barker, R. A., Bellenguez, C., Bhatia, K., Blackburn, H., Blackwell, J. M., Bramon, E., Brown, M. A., Brown, M. A., Burn, D., Casas, J. P., Chinnery, P. F., Clarke, C. E., Corvin, A., Craddock, N., Deloukas, P., Edkins, S., Evans, J., Freeman, C., Gray, E., Hardy, J., Hudson, G., Hunt, S., Jankowski, J., Langford, C., Lees, A. J., Markus, H. S., Mathew, C. G., McCarthy, M. I., Morrison, K. E., Palmer, C. N., Pearson, J. P., Peltonen, L., Pirinen, M., Plomin, R., Potter, S., Rautanen, A., Sawcer, S. J., Su, Z., Trembath, R. C., Viswanathan, A. C., Williams, N. W., Morris, H. R., Donnelly, P. & Wood, N. W. Dissection of the genetics of Parkinson's disease identifies an additional association 5′ of SNCA and multiple associated haplotypes at 17q21. Hum Mol Genet 20, 345-353 (2011).
7 Seto-Salvia, N., Clarimon, J., Pagonabarraga, J., Pascual-Sedano, B., Campolongo, A., Combarros, O., Mateo, J. I., Regana, D., Martinez-Corral, M., Marquie, M., Alcolea, D., Suarez-Calvet, M., Molina-Porcel, L., Dols, O., Gomez-Isla, T., Blesa, R., Lleo, A. & Kulisevsky, J. Dementia risk in Parkinson disease: disentangling the role of MAPT haplotypes. Arch Neurol 68, 359-364 (2011).
8 Myers, A. J., Kaleem, M., Marlowe, L., Pittman, A. M., Lees, A. J., Fung, H. C., Duckworth, J., Leung, D., Gibson, A., Morris, C. M., de Silva, R. & Hardy, J. The Hic haplotype at the MAPT locus is associated with Alzheimer's disease. Hum Mol Genet 14, 2399-2404, doi:10.1093/hmg/ddi241 (2005).
9 Pastor, P., Moreno, F., Clarimon, J., Ruiz, A., Combarros, O., Calero, M., Lopez de Munain, A., Bullido, M. J., de Pancorbo, M. M., Carro, E., Antonell, A., Coto, E., Ortega-Cubero, S., Hernandez, I., Tarraga, L., Boada, M., Lleo, A., Dols-Icardo, O., Kulisevsky, J., Vazquez-Higuera, J. L., Infante, J., Rabano, A., Fernandez-Blazquez, M. A., Valenti, M., Indakoetxea, B., Barandiaran, M., Gorostidi, A., Frank-Garcia, A., Sastre, I., Lorenzo, E., Pastor, M. A., Elcoroaristizabal, X., Lennarz, M., Maier, W., Ramirez, A., Serrano-Rios, M., Lee, S. E., Sanchez-Juan, P. & Dementia Genetic Spanish, C. MAPT H1 Haplotype is Associated with Late-Onset Alzheimer's Disease Risk in APOEvarepsilon4 Noncarriers: Results from the Dementia Genetics Spanish Consortium. J Alzheimers Dis 49, 343-352 (2016).
10 Kauwe, J. S., Cruchaga, C., Mayo, K., Fenoglio, C., Bertelsen, S., Nowotny, P., Galimberti, D., Scarpini, E., Morris, J. C., Fagan, A. M., Holtzman, D. M. & Goate, A. M. Variation in MAPT is associated with cerebrospinal fluid tau levels in the presence of amyloid-beta deposition. Proc Natl Acad Sci USA 105, 8050-8054, doi:10.1073/pnas.0801227105 (2008).
11 Allen, M., Kachadoorian, M., Quicksall, Z., Zou, F., Chai, H. S., Younkin, C., Crook, J. E., Pankratz, V. S., Carrasquillo, M. M., Krishnan, S., Nguyen, T., Ma, L., Malphrus, K., Lincoln, S., Bisceglio, G., Kolbert, C. P., Jen, J., Mukherjee, S., Kauwe, J. K., Crane, P. K., Haines, J. L., Mayeux, R., Pericak-Vance, M. A., Farrer, L. A., Schellenberg, G. D., Parisi, J. E., Petersen, R. C., Graff-Radford, N. R., Dickson, D. W., Younkin, S. G. & Ertekin-Taner, N. Association of MAPT haplotypes with Alzheimer's disease risk and MAPT brain gene expression levels. Alzheimers Res Ther 6, 39 (2014).
12 Caffrey, T. M., Joachim, C., Paracchini, S., Esiri, M. M. & Wade-Martins, R. Haplotype-specific expression of exon 10 at the human MAPT locus. Hum Mol Genet 15, 3529-3537 (2006).
13 Duff, K., Knight, H., Refolo, L. M., Sanders, S., Yu, X., Picciano, M., Malester, B., Hutton, M., Adamson, J., Goedert, M., Burki, K. & Davies, P. Characterization of pathology in transgenic mice over-expressing human genomic and cDNA tau transgenes. Neurobiol Dis 7, 87-98 (2000).
14 McMillan, P., Korvatska, E., Poorkaj, P., Evstafjeva, Z., Robinson, L., Greenup, L., Leverenz, J., Schellenberg, G. D. & D'Souza, I. Tau isoform regulation is region- and cell-specific in mouse brain. J Comp Neurol 511, 788-803 (2008).
15 Osoegawa, K., Mammoser, A. G., Wu, C., Frengen, E., Zeng, C., Catanese, J. J. & de Jong, P. J. A bacterial artificial chromosome library for sequencing the complete human genome. Genome Res 11, 483-496 (2001).
16 Raz, N., Lindenberger, U., Rodrigue, K. M., Kennedy, K. M., Head, D., Williamson, A., Dahle, C., Gerstorf, D. & Acker, J. D. Regional brain changes in aging healthy adults: general trends, individual differences and modifiers. Cereb Cortex 15, 1676-1689 (2005).
17 Grady, C. L., McIntosh, A. R., Horwitz, B., Maisog, J. M., Ungerleider, L. G., Mentis, M. J., Pietrini, P., Schapiro, M. B. & Haxby, J. V. Age-related reductions in human recognition memory due to impaired encoding. Science 269, 218-221 (1995).
18 Yassa, M. A., Lacy, J. W., Stark, S. M., Albert, M. S., Gallagher, M. & Stark, C. E. Pattern separation deficits associated with increased hippocampal CA3 and dentate gyrus activity in nondemented older adults. Hippocampus 21, 968-979, doi:10.1002/hipo.20808 (2011).
19 Silva, M. C., Cheng, C., Mair, W., Almeida, S., Fong, H., Biswas, M. H., Zhang, Z., Huang, Y., Temple, S., Coppola, G., Geschwind, D. H., Karydas, A., Miller, B. L., Kosik, K. S., Gao, F. B., Steen, J. A. & Haggarty, S. J. Human iPSC-Derived Neuronal Model of Tau-A152T Frontotemporal Dementia Reveals Tau-Mediated Mechanisms of Neuronal Vulnerability. Stem Cell Reports 7, 325-340 (2016).
20 Beevers, J. E., Lai, M. C., Collins, E., Booth, H. D. E., Zambon, F., Parkkinen, L., Vowles, J., Cowley, S. A., Wade-Martins, R. & Caffrey, T. M. MAPT genetic variation and neuronal maturity alter isoform expression affecting axonal transport in iPSC-derived dopamine neurons. Stem Cell Reports (in press).
21 Trabzuni, D., Wray, S., Vandrovcova, J., Ramasamy, A., Walker, R., Smith, C., Luk, C., Gibbs, J. R., Dillman, A., Hernandez, D. G., Arepalli, S., Singleton, A. B., Cookson, M. R., Pittman, A. M., de Silva, R., Weale, M. E., Hardy, J. & Ryten, M. MAPT expression and splicing is differentially regulated by brain region: relation to genotype and implication for tauopathies. Hum Mol Genet 21, 4094-4103 (2012).
22 Dubose, A. J., Lichtenstein, S. T., Narisu, N., Bonnycastle, L. L., Swift, A. J., Chines, P. S. & Collins, F. S. Use of microarray hybrid capture and next-generation sequencing to identify the anatomy of a transgene. Nucleic Acids Res 41, e70 (2013).
23 Benzow, K. A. & Koob, M. D. Markerless modification of trinucleotide repeat loci in BACs. Methods Mol Biol 1010, 265-276 (2013).
24 Beard, C., Hochedlinger, K., Plath, K., Wutz, A. & Jaenisch, R. Efficient method to generate single-copy transgenic mice by site-specific integration in embryonic stem cells. Genesis 44, 23-28 (2006).
25 Livet, J., Weissman, T. A., Kang, H., Draft, R. W., Lu, J., Bennis, R. A., Sanes, J. R. & Lichtman, J. W. Transgenic strategies for combinatorial expression of fluorescent proteins in the nervous system. Nature 450, 56-62 (2007).
26 Ingram, M., Wozniak, E. A. L., Duvick, L., Yang, R., Bergmann, P., Carson, R., O'Callaghan, B., Zoghbi, H. Y., Henzler, C. & Orr, H. T. Cerebellar Transcriptome Profiles of ATXN1 Transgenic Mice Reveal SCA1 Disease Progression and Protection Pathways. Neuron 89, 1194-1207 (2016).
27 de Silva, R., Lashley, T., Gibb, G., Hanger, D., Hope, A., Reid, A., Bandopadhyay, R., Utton, M., Strand, C., Jowett, T., Khan, N., Anderton, B., Wood, N., Holton, J., Revesz, T. & Lees, A. Pathological inclusion bodies in tauopathies contain distinct complements of tau with three or four microtubule-binding repeat domains as demonstrated by new specific monoclonal antibodies. Neuropathol Appl Neurobiol 29, 288-302 (2003).
28 Jicha, G. A., Bowser, R., Kazam, I. G. & Davies, P. Alz-50 and MC-1, a new monoclonal antibody raised to paired helical filaments, recognize conformational epitopes on recombinant tau. J Neurosci Res 48, 128-132 (1997).
29 Herskovits, A. Z. & Davies, P. The regulation of tau phosphorylation by PCTAIRE 3: implications for the pathogenesis of Alzheimer's disease. Neurobiol Dis 23, 398-408 (2006).
30 Mercken, M., Vandermeeren, M., Lubke, U., Six, J., Boons, J., Van de Voorde, A., Martin, J. J. & Gheuens, J. Monoclonal antibodies with selective specificity for Alzheimer Tau are directed against phosphatase-sensitive epitopes. Acta Neuropathol 84, 265-272 (1992).
31 Goedert, M., Jakes, R. & Vanmechelen, E. Monoclonal antibody AT8 recognises tau protein phosphorylated at both serine 202 and threonine 205. Neurosci Lett 189, 167-169 (1995).
32 Goedert, M., Jakes, R., Crowther, R. A., Cohen, P., Vanmechelen, E., Vandermeeren, M. & Cras, P. Epitope mapping of monoclonal antibodies to the paired helical filaments of Alzheimer's disease: identification of phosphorylation sites in tau protein. Biochem J 301 (Pt 3), 871-877 (1994).
33 Min, S. W., Cho, S. H., Zhou, Y., Schroeder, S., Haroutunian, V., Seeley, W. W., Huang, E. J., Shen, Y., Masliah, E., Mukherjee, C., Meyers, D., Cole, P. A., Ott, M. & Gan, L. Acetylation of tau inhibits its degradation and contributes to tauopathy. Neuron 67, 953-966 (2010).
34 Zhao, X., Kotilinek, L. A., Smith, B., Hlynialuk, C., Zahs, K., Ramsden, M., Cleary, J. & Ashe, K. H. Caspase-2 cleavage of tau reversibly impairs memory. Nat Med 22, 1268-1276 (2016).
35 Rissman, R. A., Poon, W. W., Blurton-Jones, M., Oddo, S., Torp, R., Vitek, M. P., LaFerla, F. M., Rohn, T. T. & Cotman, C. W. Caspase-cleavage of tau is an early event in Alzheimer disease tangle pathology. J Clin Invest 114, 121-130 (2004).
36 de Leon, M. J., Segal, S., Tarshish, C. Y., DeSanti, S., Zinkowski, R., Mehta, P. D., Convit, A., Caraos, C., Rusinek, H., Tsui, W., Saint Louis, L. A., DeBernardis, J., Kerkman, D., Qadri, F., Gary, A., Lesbre, P., Wisniewski, T., Poirier, J. & Davies, P. Longitudinal cerebrospinal fluid tau load increases in mild cognitive impairment. Neurosci Lett 333, 183-186 (2002).
37 Eng, L. F. Glial fibrillary acidic protein (GFAP): the major protein of glial intermediate filaments in differentiated astrocytes. J Neuroimmunol 8, 203-214 (1985).
38 Ito, D., Imai, Y., Ohsawa, K., Nakajima, K., Fukuuchi, Y. & Kohsaka, S. Microglia-specific localisation of a novel calcium binding protein, Iba1. Brain Res Mol Brain Res 57, 1-9 (1998).
39 Crary, J. F., Trojanowski, J. Q., Schneider, J. A., Abisambra, J. F., Abner, E. L., Alafuzoff, I., Arnold, S. E., Attems, J., Beach, T. G., Bigio, E. H., Cairns, N. J., Dickson, D. W., Gearing, M., Grinberg, L. T., Hof, P. R., Hyman, B. T., Jellinger, K., Jicha, G. A., Kovacs, G. G., Knopman, D. S., Kofler, J., Kukull, W. A., Mackenzie, I. R., Masliah, E., McKee, A., Montine, T. J., Murray, M. E., Neltner, J. H., Santa-Maria, I., Seeley, W. W., Serrano-Pozo, A., Shelanski, M. L., Stein, T., Takao, M., Thal, D. R., Toledo, J. B., Troncoso, J. C., Vonsattel, J. P., White, C. L., 3rd, Wisniewski, T., Woltjer, R. L., Yamada, M. & Nelson, P. T. Primary age-related tauopathy (PART): a common pathology associated with human aging. Acta Neuropathol 128, 755-766 (2014).
40 Hsiao, K., Chapman, P., Nilsen, S., Eckman, C., Harigaya, Y., Younkin, S., Yang, F. & Cole, G. Correlative memory deficits, Abeta elevation, and amyloid plaques in transgenic mice. Science 274, 99-102 (1996).
41 Mair, W., Muntel, J., Tepper, K., Tang, S., Biernat, J., Seeley, W. W., Kosik, K. S., Mandelkow, E., Steen, H. & Steen, J. A. FLEXITau: Quantifying Post-translational Modifications of Tau Protein in Vitro and in Human Disease. Anal Chem 88, 3704-3714 (2016).
42 Avivi-Arber, L., Seltzer, Z., Friedel, M., Lerch, J. P., Moayedi, M., Davis, K. D. & Sessle, B. J. Widespread Volumetric Brain Changes following Tooth Loss in Female Mice. Front Neuroanat 10, 121 (2016).
43 Saito, T., Matsuba, Y., Mihira, N., Takano, J., Nilsson, P., Itohara, S., Iwata, N. & Saido, T. C. Single App knock-in mouse models of Alzheimer's disease. Nat Neurosci 17, 661-663 (2014).

### Example 3—Moving Human Genetics into the Mouse: Full Human Gene-Replacement Models

Although geneticists have now identified many of the sequence variants that underlie a wide array of human diseases, most of the animal models we have made in light of these findings have failed to capture more than a fraction of the molecular impacts of these pathogenic variants. In the first public presentation of this work, we report that we have developed Gene Replacement (GR) technology that allows us to more fully mimic the genetics of human disease in mice by replacing mouse genes with their full human orthologs. We used this technology to replace the full mouse Microtubule-associated protein tau (Mapt) genomic coding and regulatory region (156,547 bp) with the full human MAPT genomic sequence (190,081 bp). We have confirmed that mice homozygous for this MAPT-GR allele express human tau at endogenous levels, and that all expected splice variants are found in the appropriate tissues and in ratios expected for the fully functional human MAPT gene. The genomes of all subsequent models generated in this MAPT-GR series of mouse lines will precisely match our first MAPT-GR line except for those MAPT nucleotide differences that we specifically introduce. These matched sets of animal models will allow the research community to evaluate the molecular impact of pathogenic mutations within the context of the human genomic sequence in which they occur in patients, and these mouse lines will contain all potential human therapeutic targets of the MAPT gene. Furthermore, by fully replacing the endogenous Mapt gene with its human counterpart, a) the MAPT gene in these lines is situated in the only location within the mouse genome that has specifically evolved to express the tau gene, and b) the MAPT-GR lines do not express any endogenous mouse tau gene products that could confound analyses of these mice. We are working to generate similar lines in which other human genes involved in the etiology of Alzheimer's Disease (AD) replace their mouse homologs, and will discuss our progress in characterizing our first lines with a 356 kb Amyloid Precursor Protein (APP)-GR allele. Finally, to demonstrate the utility of these approaches to diseases other than AD, we will describe matched sets of partial Gene Replacements (pGR) lines with either 31 kb ATXN1-pGR alleles (wt+5 matched SpinoCerebellar Ataxia type 1 variants) or 192 kb TCF4-pGR alleles (wt+2 matched Fuchs Endothelial Corneal Dystrophy CTG expansion mutations).

Results

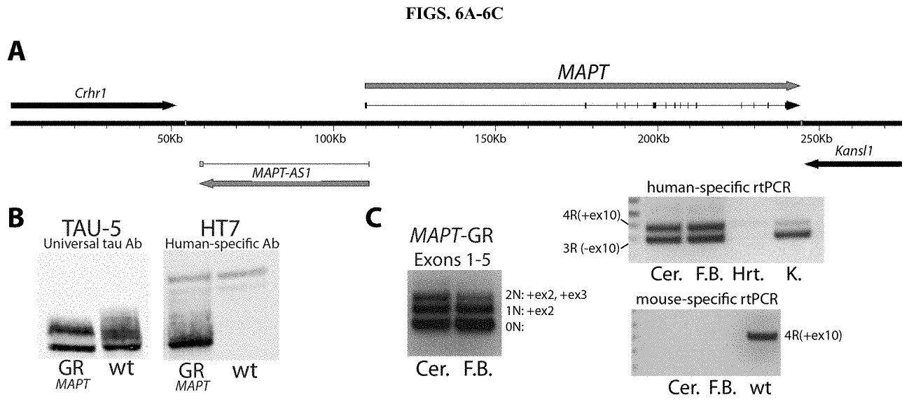

FIG. 6A illustrates precise replacement of the 157 kb mouse Mapt gene with the 190 kb human syntenic genomic segment encoding the complete MAPT coding region and MAPT-AS1 regulatory region (in green), as confirmed by NGS whole genome sequencing of mice homozygous for this MAPT-Gene Replacement (MAPT-GR) allele. The exons in the MAPT mRNA and in the long-noncoding antisense RNA transcripts are indicated, and flanking mouse genes are shown in black (size scale in kilobases). As demonstrated in FIG. 6B, Western analyses show that human tau protein is expressed at endogenous levels in MAPT-GR mice. Equal amounts (50 μg) of protein isolated from the forebrain tissue of either a mouse homozygous for the transplanted MAPT-GR allele (GR) or from a wt C57BL/6 mouse (wt) were analyzed with an antibody (TAU-5) that binds equally well to either human or mouse tau protein. The equal intensity of signal from both sample shows that the human tau protein is expressed at levels equivalent to that of mouse tau. Analyses with an antibody that only recognizes human tau demonstrate that the tau expressed in the MAPT-GR mice is human (the bands in the mouse lane are non-tau background recognized by this antibody, also present in the MAPT-GR sample). Transcription analyses show that all of the human MAPT splice isoforms are expressed in the tissues and in the ratios expected for the human MAPT gene (FIG. 6C). RNA isolated from the cerebellum (Cer.) forebrain (F.B.), heart (Hrt.), and kidney (K.) of MAPT-GR homozygous mice, as well as from the forebrain of a wt C57BL/6 control animal (wt) were analyzed by rtPCR. We found MAPT transcripts that encoded either “0N” (no exon 2 or 3), “1N” (exon 2 but not 3), and “2N” (both exon 2 and 3) tau (first panel). We also found roughly equal levels of “3R” (no exon 10) and “4R” (with exon 10) MAPT transcripts in brain tissues, with different ratios in the other tissues examined, as is typical for the human MAPT gene (human-specific rtPCR panel). No mouse Mapt transcripts were detected in the MAPT-GR homozygote samples using mouse-specific rtPCR, but the expected predominance of “4R” mouse tau transcript was found in the forebrain sample from the adult wt control animal.

Materials and Methods

DNA Constructs:

Gene specific homology was added to corresponding target ends from assembled generic plasmids.

Mapt-Kans1 FRT3 CBh tdTomato NoBstGI loxP T2A Cre 1xT L-FRT Hygo LoxN

### -OR-

Mapt-Kans1 FRT3 CBh tdTomato NoBstGI loxP T2A Cre 2xT L-FRT Hygo LoxN

### Mapt-AS1 Arm2 LoxP T2A Puro WPRESv4OTT FRT3 Arm1 pBS (+) ori

It was observed that the FRT sites could be replaced by Bxb1 attP/attB recombination sites or PhiC31 attP/attB recombination sites in this protocol.

Bxb1 attB:
(SEQ ID NO: 6)
TCGGCCGGCTTGTCGACGACGGCGGTCTCCGTCGTCAGGATCATCCGGGC
Bxb1 attP:
(SEQ ID NO: 7)
GTCGTGGTTTGTCTGGTCAACCACCGCGGTCTCAGTGGTGTACGGTACAA
ACCCCGAC

Bxb1 attP and attB recombine to form Bxb1 attL and attR:

Bxb1 attL:
(SEQ ID NO: 8)
TCGGCCGGCTTGTCGACGACGGCGGTCTCAGTGGTGTACGGTACAAACCC
CGAC
Bxb1 attR:
(SEQ ID NO: 9)
GTCGTGGTTTGTCTGGTCAACCACCGCGGTCTCCGTCGTCAGGATCATCC
GGGC
PhiC31 attB:
(SEQ ID NO: 10)
TGCGGGTGCCAGGGCGTGCCCTTGGGCTCCCCGGGCGCGTACTCC
PhiC31 attP:
(SEQ ID NO: 11)
GTGCCCCAACTGGGGTAACCTTTGAGTTCTCTCAGTTGGGGG

PhiC31 attP and attB recombine to form PhiC31 attL and attR:

PhiC31 attL:
(SEQ ID NO: 12)
TGCGGGTGCCAGGGCGTGCCCTTGAGTTCTCTCAGTTGGGGG
PhiC31 attR:
(SEQ ID NO: 13)
GTGCCCCAACTGGGGTAACCTTTGGGCTCCCCGGGCGCGTACTCC

CRISPR Constructs:

sgRNA targets were designed for flanking the gene or gene segment (Mouse Tau) of interest and cloned into the pSpCas9(BB)-2A-Puro (Addgene plasmid ID 48139) as per published protocols (Nature Protocols Vol. 8 No. 11: 2281-2308 (2013)).

pSpCas9(BB)-2A-Puro Mapt B sgRNA
pSpCas9(BB)-2A-Puro Kans1 B sgRNA

Embryonic Stem Cell Culture:

C57BL/6N-PRX-B6N mouse embryonic stem (ES) cells where purchased from Jax Laboratory (Donating Investigator Robin Wesselschmidt, Primogenix, Inc). Cells were maintained at 37° C. at 7.5% CO₂ in IMDM (Iscove's DMEM) media containing 20% ESC qualified FBS, 1× NEAA, 2 mM L-glutamine, 1× Penicillin/Streptomycin, 0.2 mM Beta-Mercaptoethanol, 1000 U/mL ESGRO-mouse LIF. Cells were grown on gelatin coated dishes containing irradiated mouse embryonic fibroblasts (iMEF).

KO Transfections, Selection, Analysis and Expansion:

C57BL/6N-PRX-B6N mES cells were trypsinized with 0.05% Trypsin for 5 minutes in the early am and plated onto gelatin coated (no iMEF) 6 well dishes from a 90% ESC confluent p60 dish at a 1:6 dilution. 3 μg of each KO target arm plasmid and 1 μg of each sgRNA targeted pSpCAS9(BB)-2A-Puro plasmid, (4 plasmids total) were co-mingle all day. When the ES cells attached to the dish (late afternoon), 32 μl of 1 mg/mL 25 kDa linear polyethylenimine (PEI) (Polysciences, Inc.) was added to 500 uL of OptiMEM. The 4 plasmid co-mingle DNA mixture was added to 500 mL of OptiMEM. The PEI/OptiMEM mixture was then added to the DNA mixture, gently vortexed and incubated at room temperature for 30 minutes. The transfection complex (1 mL) was dropped onto ES cells in 6 well dishes with 2 mL of fresh complete media for an overnight incubation with conditions above. The next day, ESC were transferred to 10 cm, gelatin coated iMEF-puroR plates. Cells were allowed to recover for 24 hours. 1.4 μg/mL Puromycin was added on day 2 and 3. No Puromycin on day 4. 1.5 ug/mL Puromycin was added on days 5-7. On day 8, clones were picked onto 96 well gelatin, iMEF plates. When cells were 80-90% confluent, ES cells were frozen in duplicate in 96 well format (80% complete media, 10% additional ES-FBS, 10% DMSO), and a 48 well gelatin only plate was allowed to expand and harvested for DNA analysis of each clone.

All junctions were analyzed by PCR and subsequent sequencing. WT assays were also performed. If the Cre mediated KO area was successful, the ES cells will now fluoresce tomato.

Junction Analysis Primers (Tau):

| | |
|---|---|
| Middle Junction: | PuroR rev assay: 5′ GAGTTCTTGCAGCTCGGTGA 3′ (SEQ ID NO: 14) |
| | td Tomato 3′ Rev out: 5′ GGCATGGACGAGCTGTATA 3′(SEQ ID |
| | NO: 15) |
| MAPT Junction: | F2 MAPT AS: 5′ GGACTGAGGTGGGGGTGATA 3′ (SEQ ID NO: 16) |
| | SV40 Poly A Rev: 5′ GCTTTATTTGTGAAATTTGTGATGC 3′ (SEQ |
| | ID NO: 17) |
| KANSL Junction: | Kansl Insertion Assay R1: 5′ GCACATCATCTGGGAGAGACTA 3′ |
| | (SEQ ID NO: 18) |
| | Hygro to 3′ arm F2: 5′ CAGACGCGCTACTTCGAGC 3′ (SEQ ID |
| | NO: 19) |
| WT assays: | |
| KANSL end: | MAPT-KANSL F: 5′ TAGGTTTGCAGAGCTGCCTT 3′ (SEQ ID |
| | NO: 25) |
| | Kansl Insertion Assay R1: 5′ GCACATCATCTGGGAGAGACTA 3′ |
| | (SEQ ID NO: 20) |

Positive clones were expanded from 96 well dishes to multiple p60's and frozen. Karyotype analysis was also done.

Large Scale Preparation of BAC DNA:

Qiagen EndoFree Mega Plasmid Kit Cat No./ID: 12381. NucleoBond folded filters XL (Takara Bio USA, ref #740577) were used in place of the plunger or vacuum filtration supplied with the Qiagen kit.

KI Transfection, Selection, Analysis and Expansion:

KO C57B6 ES clone (1XTC22) was cultured to 80% confluency on p60 gelatin coated, iMef dishes. The morning of transfection, ES cells (ESCs) were trypsinized (0.05%) for 5 minutes. ES cells were re-plated onto gelatin only, 6 well dishes at 1:6 dilution. Cells were allowed to attach to the dish prior to transfection in late afternoon. 6 μg of the human Tau+LoxN BAC and 2 μg of pCAGGS FlpE recombinase were co-transfected with 32 μl of 1 μg/μl 25K linear PEI in 1 mL of OptiMEM. After a 30-minute room temperature incubation, the transfection complex (1 mL) was dropped onto the 1XTC22 KO ES cells in 6 well dishes with 2 mL of fresh complete media for an overnight incubation at 37° C., 6.5% CO₂. The next day, ES cells were transferred to 10 cm, gelatin coated iMEF-HygroR plates. Cells were allowed to recover for 48 hours. Cells were then selected with 60 μg/ml hygromycin for 7 days. 1 positive clone was picked and expanded to multiple p60's. The H2 Tau KI clone was frozen in 80% complete media, 10% additional ES-FBS, 10% DMSO.

DNA Analysis:

F2 MAPT AS: 5′ GGACTGAGGTGGGGGTGATA 3′ (SEQ ID NO: 21)
3′ of MAPT -AS assay: 5′ AGTCAGTTGGCAGTTTGGATGA 3′ (SEQ ID NO: 22)
Primer B Assay: 5′ ACGAGCCTTCATAGCATCCG 3′ (SEQ ID NO: 23)
Hygro connect: 5′ ATCCACGCCCTCCTACATCGAA 3′ (SEQ ID NO: 24)
Internal H2 Tau
PmeI MAPT F
PmeI MAPT R
Primer B Rev
3′ KANSL 1 assay
413p22 end F
413p22 end R
Neg BAC only assay (off target integration)
5′ BAC CmR
3′ of KANSL assay

Clone 1 was correct for all assays and was further expanded and chimeric mice were generated by injection into blastocysts. Positive F1's were crossed to Sox Cre mice to remove the selectable marker and further backcrossed with C57B6/J, and these heterozygous mice were crossed to obtain mice homozygous for the human MAP T-GR allele.

Discussion

By incorporating full human genes in place of their mouse homolog, the Gene Replacement mouse models that we are generating represent a substantial departure from the status quo of currently available models. Construction of these innovative models is made possible by the GR technology we have developed that allows us to routinely replace mouse genes with their full human orthologs up to several hundred kb in size. The standard tools of molecular genetics are still best adapted for working with relatively small DNA fragments. As a direct result of these technical limitations, most of the animal models of human disease generated by researchers and currently available to the wider biomedical research community incorporate short, synthetic and very simplified cDNA versions of genes expressed from short, exogenous promoter fragments, These cDNA alleles can be powerful tools for testing specific hypotheses and for focusing on defined aspects of a gene's function, but they are typically not capable of capturing more than a fraction of the subtle complexity of a full human gene. Although significant strides are being made towards generating the next generation of mouse models by precisely modifying full genes, technical limitations have in most instances restricted these efforts to making relatively short sequence modifications to the endogenous mouse genes rather than replacing them with their full human ortholog.

### Example 4—Producing an Amyloid Beta Precursor Protein Gene Replacement (APP-GR) Mouse Comprising the Human APP Gene

Amyloid precursor protein (APP) is the precursor molecule that is cleaved to generate beta amyloid peptides. Beta amyloid is a component of amyloid plaques associated with Alzheimer's disease (AD). AD is the most common neurodegenerative disease affecting 60-80% of all dementia cases. There is no cure for AD, as such the study of this disease is an active area of basic and translational research. There are several animal models of AD, each with its distinct advantages and disadvantages.

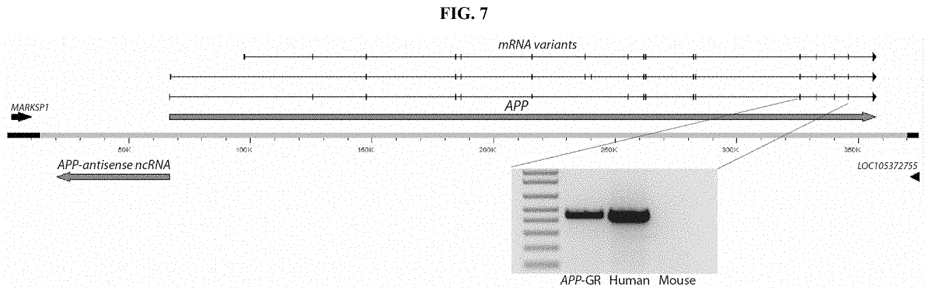

The methods of this disclosure were performed to obtain to mice carrying APP-GR alleles, in which a 290 kb segment of the mouse genome encoding the App gene is replaced with a syntenic 356 kb APP sense/antisense human genomic segment. Initial results demonstrate that the complete sequence of the human genomic construct used to generate these new engineered mouse lines is correct, and that the expected junctions between the mouse and human genomic segment were obtained. While complete genome analyses of these lines to confirm the full integrity of the APP-GR allele are ongoing, sacrifice of one of the chimeric animals that could not be used for breeding (a female mouse made from male ES cells) revealed that the amyloid beta (Aβ) portion of the human APP gene included in these human-specific rtPCR analyses was transcribed and spliced as expected (FIG. 7). Accordingly, data obtained to date are consistent with replacement of the mouse App gene with a syntenic human genomic segment. Mice comprising the full-length human APP gene can be further genetically modified to introduce clinically relevant mutations associated with various disease phenotypes. Accordingly, an APP gene replacement (APP-GR) mouse produced according to the methods of this disclosure will provide valuable tool for basic and clinical trial research since it allows the genetic modification of the human transgene to achieve a particular phenotype.

It is to be understood that the above description is intended to be illustrative, and not restrictive. The present disclosure has described one or more preferred embodiments, and it should be appreciated that many equivalents, alternatives, variations, and modifications, aside from those expressly stated, are possible and within the scope of the invention. Other embodiments will be apparent to those of skill in the art upon reading and understanding the above description. It should be noted that embodiments discussed in different portions of the description or referred to in different drawings can be combined to form additional embodiments of the present application. The scope should, therefore, be determined with reference to the appended claims, along with the full scope of equivalents to which such claims are entitled.

We claim:

1. A method for removing an endogenous gene in the genome of a non-human animal, the method comprising:
introducing into a non-human animal cell:
(a) a first construct comprising (i) a nucleic acid sequence encoding a first selectable marker, the nucleic acid sequence flanked by a first recombination site and a second recombination site, (ii) a promoter, and (iii) a nucleic acid sequence encoding a first recombinase, wherein components (ii) and (iii) are separated by a third recombination site, wherein the first, second, and third recombination sites do not recombine with each other, whereby the first selectable marker and the recombinase protein products are expressed by a single open reading frame,
(b) a second construct comprising a nucleic acid sequence encoding a second selectable marker flanked by a fourth recombination site and a fifth recombination site, wherein the fourth recombination site is upstream of the second selectable marker and is capable of recombining with the third recombination site in the presence of the first recombinase;
(c) one or more Cas/gRNA constructs comprising one or more gRNAs having sequence complementary to at least a portion of the endogenous non-human gene, and
wherein gRNAs expressed from the one or more Cas/gRNA constructs associate with the endogenous target gene and generate double stranded breaks 5′ and 3′ to the endogenous non-human gene,
wherein the double stranded breaks are repaired by homology-directed repair using the first and second constructs, whereby the first and second constructs are inserted 5′ and 3′ to the endogenous non-human target gene, and
wherein expression of the recombinase from the first construct catalyzes recombination between the third and fourth recombination sites, whereby the endogenous non-human gene and the nucleic acid encoding the recombinase are excised and the promoter drives expression of the selectable marker.

2. The method of claim 1, wherein the first targeting construct further comprises, located between the third recombination site and the first recombinase-encoding sequence, a nucleic acid sequence encoding a peptide separation linker.

3. The method of claim 1, further comprising selecting a non-human cell in which the endogenous non-human gene is excised by detecting expression of the selectable marker and the screening marker.

4. The method of claim 1, wherein the first or second selectable marker is a fluorescent marker.

5. The method of claim 1, wherein the first or second selectable marker is a drug resistance marker.

6. The method of claim 1, wherein the third and fourth recombination sites are lox recombination sites and wherein the site-specific recombinase is Cre.

7. A method for replacing an endogenous gene in the genome of a non-human animal with a syntenic sequence from a human genome, the method comprising
obtaining a non-human cell in which an endogenous gene has been excised according to the method of claim 1;
introducing into the obtained cell a nucleic acid vector comprising nucleic acid sequence syntenic to the excised endogenous gene, where the syntenic sequence is flanked on the 5′ end by a sixth recombination site and flanked on the 3′ end by a seventh recombination site, the vector further comprising a promoter operably linked to a nucleic acid sequence encoding a second recombinase and an eighth recombination site, where the fifth and seventh recombination sites recombine with each other, and where the second and eighth recombination sites recombine with each other in the presence of the second recombinase;
wherein expression of the first recombinase catalyzes recombination between the first and sixth recombination sites, resulting in insertion of the syntenic sequence in place of the excised endogenous non-human gene, and wherein expression of the second recombinase catalyzes recombination between the fifth and seventh recombination sites and the second and eighth recombination sites to excise nucleic acid sequences encoding the screening marker and the selection marker.

8. The method of claim 7, wherein the second and eighth recombination sites are FRT recombination sites, and wherein the fifth and seventh recombination sites are FRT3 recombination sites.

9. The method of claim 7, wherein the nucleic acid vector comprises a PGK promoter.

10. A genetically modified non-human cell comprising a human syntenic gene generated by the method of claim 7.

11. The non-human cell of claim 10, wherein the non-human animal cell is a mouse embryonic cell.

12. A genetically modified non-human animal generated from the cell of claim 10.

13. The genetically modified non-human animal of claim 12, wherein said animal is a mammal.

14. The genetically modified non-human animal of claim 13, wherein said animal is chosen from a mouse, a rat, a rabbit, a pig, a sheep, a goat, poultry, and a cow.
